(19) United States
(12) Patent Application Publication
(10) Pub. No.: US 2024/0349598 A1
(43) Pub. Date: Oct. 17, 2024

(54) ORGANIC COMPOUND AND ORGANIC LIGHT EMITTING DIODE COMPRISING SAME

(71) Applicant: MATERIAL SCIENCE CO.,LTD., Seoul (KR)

(72) Inventors: Ha-ni JEON, Seoul (KR); Tae-Wan LEE, Seoul (KR); Jie SONG, Seoul (KR); Seung-Hye JEONG, Seoul (KR)

(21) Appl. No.: 18/634,113

(22) Filed: Apr. 12, 2024

(30) Foreign Application Priority Data
Apr. 14, 2023 (KR) 10-2023-0049281
Apr. 9, 2024 (KR) 10-2024-0048084

(51) Int. Cl. H10K 85/60; C07D 307/91; C07D 405/12; C07D 409/12; C09K 11/06; H10K 50/15

(52) U.S. Cl. CPC H10K 85/636; C07D 307/91; C07D 405/12; C07D 409/12; C09K 11/06; H10K 85/633; C09K 2211/1018; H10K 50/15; H10K 85/615; H10K 85/626; H10K 85/6572; H10K 85/6574; H10K 85/6576

(57) ABSTRACT

The organic compound represented by chemical formula 1 of the present disclosure can implement excellent hole injection and hole transport characteristics. In addition, the hole transport auxiliary layer of the organic light emitting diode of the present disclosure can improve the driving voltage, efficiency, and lifetime characteristics of the organic light emitting diode by including the organic compound represented by Chemical Formula 1 of the present disclosure.

### CROSS REFERENCE TO RELATED APPLICATION

The present application claims priority to Korean Patent Applications No. 10-2023-0049281 filed on Apr. 14, 2023 and No. 10-2024-0048084 filed on Apr. 9, 2024, the entire contents of which is incorporated herein for all purposes by this reference.

### BACKGROUND OF THE INVENTION

### Field of the Invention

The present disclosure relates to an organic compound and an organic light emitting diode including the same.

### Description of the Related Art

Since organic light emitting diodes (OLEDs) have a simple structure, various advantages in a manufacturing process, high brightness, a wide viewing angle, a fast response time, and a low driving voltage compared to other flat panel display devices, such as current liquid crystal display (LCD) devices, plasma display panel (PDP) devices, and field emission display (FED) devices, the OLEDs are being actively developed and commercialized to be used as flat displays, such as wall-mounted TVs, or light sources, such as backlight of displays, lighting, and billboards.

The OLED is formed of an organic layer between two electrodes. The OLED is an element using a principle in which electrons and holes are injected into an emitting layer from each of the two electrodes, excitons are generated by combining electrons and holes, and when the generated excitons are transitioned from an excited state to a ground state, light is emitted.

The OLED may include at least one emitting layer. In general, an OLED with a plurality of emitting layers may include emitting layers that emit light with different peak wavelengths, thereby implementing a specific color through a combination of light with different peak wavelengths.

The OLED may be classified into a top emission element structure and a bottom emission element structure. The top emission device emits light generated from the emitting layer toward a translucent anode using a reflective cathode. On the other hand, in the bottom emission device, light is emitted from the emitting layer using the reflective anode, and the light reflected by the anode is emitted toward the transparent cathode that is a direction of a driving thin film transistor.

### DOCUMENTS OF RELATED ART

(Patent Document 1) KR 2022-0010057 A
(Patent Document 2) KR 2019-0020514 A

### SUMMARY OF THE INVENTION

The present disclosure is directed to providing a novel organic compound and an organic lighting emitting diode including the same.

In addition to the above objects, embodiments according to the present disclosure may be used to achieve other objects not specifically mentioned.

The objects of the present disclosure are not limited to the above-described object, and other objects and advantages of the present disclosure which are not mentioned can be understood by the following description and more clearly understood by embodiments of the present disclosure. In addition, it can be easily seen that the objects and advantages of the present disclosure can be achieved by means and combinations thereof which are described in the claims.

According to one aspect of the present disclosure, an organic compound represented by Chemical Formula 1 below may be provided.

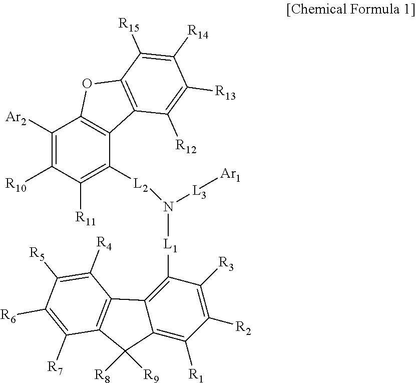

in Chemical Formula 1,

L₁ to L₃ are, each independently, selected from the group consisting of a single bond; a substituted or unsubstituted aryl group having 6 to 30 carbon atoms; a substituted or unsubstituted aralkyl group having 7 to 30 carbon atoms; a substituted or unsubstituted heteroaryl group having 5 to 60 carbon atoms; and a substituted or unsubstituted heteroarylalkyl group having 6 to 60 carbon atoms,

R₁ to R₁₅, Ar₁ and Ar₂ are, each independently, selected from the group consisting of hydrogen; deuterium; a cyano group; a nitro group; a halogen group; a hydroxy group; a substituted or unsubstituted alkyl group having 1 to 30 carbon atoms; a substituted or unsubstituted alkenyl group having 2 to 30 carbon atoms; a substituted or unsubstituted alkynyl group having 2 to 24 carbon atoms; a substituted or unsubstituted heteroalkyl group having 2 to 30 carbon atoms; a substituted or unsubstituted aryl group having 6 to 30 carbon atoms; a substituted or unsubstituted heteroaryl group having 2 to 30 carbon atoms; a substituted or unsubstituted heteroarylalkyl group having 3 to 30 carbon atoms; a substituted or unsubstituted aralkyl group having 6 to 30 carbon atoms; a substituted or unsubstituted cycloalkyl group having 3 to 20 carbon atoms; a substituted or unsubstituted heterocycloalkyl group having 3 to 20 carbon atoms; a substituted or unsubstituted alkoxy group having 1 to 30 carbon atoms; a substituted or unsubstituted alkylsilyl group having 1 to 30 carbon atoms; a substituted or unsubstituted arylsilyl group having 6 to 30 carbon atoms; and a substituted or unsubstituted aryloxy group having 6 to 30 carbon atoms, and

optionally, when L₁ to L₃, R₁ to R₁₅, and Ar₁ and Ar₂ are substituted, substituents may be one or more selected from the group consisting of deuterium; a cyano group; a nitro group; halogen; a hydroxy group; an alkyl group having 1 to 30 carbon atoms; an alkenyl group having 2 to 30 carbon atoms; an alkynyl group having 2 to 24 carbon atoms; a heteroalkyl group having 2 to 30 carbon atoms; an aralkyl group having 6 to 30 carbon atoms; a cycloalkyl group having 3 to 20 carbon atoms; a heterocycloalkyl group having 3 to 20 carbon atoms; an aryl group having 6 to 30 carbon atoms; a heteroaryl group having 2 to 30 carbon atoms; a heteroarylalkyl group having 3 to 30 carbon atoms; an alkoxy group having 1 to 30 carbon atoms; an alkylsilyl group having 1 to 30 carbon atoms; an arylsilyl group having 6 to 30 carbon atoms; and an aryloxy group having 6 to 30 carbon atoms; and when a plurality of substituents are present, each substituent is the same as or different from each other.

According to another aspect of the present disclosure, there may be provided an organic light emitting diode including an anode, a cathode facing the anode, and one or more organic layers between the anode and the cathode, wherein at least one of the organic layers includes an organic compound represented by Chemical Formula 1.

The organic compound represented by chemical formula 1 of the present disclosure can implement excellent hole injection and hole transport characteristics.

In addition, the hole transport auxiliary layer of the organic light emitting diode of the present disclosure can improve the driving voltage, efficiency, and lifetime characteristics of the organic light emitting diode by including the organic compound represented by Chemical Formula 1 of the present disclosure.

In addition, when the organic compound represented by Chemical Formula 1 of the present disclosure is used, the organic compound may have the suitable energy level as the hole transport auxiliary layer that serves to transmit holes from the hole transport layer to the emitting layer and block electrons coming from the emitting layer.

In addition, the organic light emitting diode of the present disclosure can implement excellent color coordinates targeted by the emitting layer even when the hole transport auxiliary layer including the organic compound represented by Chemical Formula 1 of the present disclosure is combined with the emitting layer of any color.

The effects of the present disclosure are not limited to the above-described effects, and other effects that are not mentioned will be able to be clearly understood by those skilled in the art from the overall description of the specification.

### DETAILED DESCRIPTION OF THE INVENTION

The above-described objects, features, and advantages will be described below in detail with reference to the following embodiments, and thus those skilled in the art to which the present disclosure pertains will be able to easily carry out the technical spirit of the present disclosure.

In describing the present specification, when it is determined that the detailed description of a related known technology may unnecessarily obscure the gist of the present specification, detailed description thereof will be omitted.

In the present specification, when terms “including” “containing”, “having”, “consisting of”, “arranging”, “providing”, and the like are used, other portions can be added unless “˜only” is used. When a component is expressed in the singular, it includes a case in which the component is provided as a plurality of components unless specifically stated otherwise.

In construing a component in the present specification, the component is construed as including the margin of error even when there is no separate explicit description.

In the present specification, the arrangement of an arbitrary component on an “upper portion (or a lower portion)” of a component or “above (or under)” the component may not only mean that the arbitrary component is disposed in contact with an upper surface (or a lower surface) of the component, but also mean that other components may be interposed between the component and the arbitrary component disposed above (or under) the component.

The term “halogen group” used herein includes fluorine, chlorine, bromine, and iodine.

The term “alkyl group” used herein indicates both linear alkyl radicals and branched alkyl radicals. Unless otherwise stated, the alkyl group contains 1 to 10 carbon atoms and may include methyl, ethyl, propyl, isopropyl, butyl, secondary butyl, isobutyl, tert-butyl, pentyl, isoamyl, hexyl, and the like, but is not limited thereto. Additionally, the alkyl group may be substituted arbitrarily.

The term “cycloalkyl group” used herein indicates cyclic alkyl radicals. Unless otherwise stated, the cycloalkyl group contains 3 to 10 carbon atoms and may include cyclopropyl, cyclobutyl, cyclopentyl, cyclohexyl, norbornyl, adamantyl, and the like, but is not limited thereto. Additionally, the cycloalkyl group may be substituted arbitrarily.

The term “alkenyl group” used herein indicates both linear alkenyl radicals and branched alkenyl radicals that have one or more carbon-carbon double bonds. Unless otherwise stated, the alkenyl group contains 2 to 10 carbon atoms and may include vinyl, allyl, isopropenyl, 2-butenyl, and the like, but is not limited thereto. Additionally, the alkenyl group may be substituted arbitrarily.

The term “cycloalkenyl group” used herein indicates cyclic alkenyl radicals. Unless otherwise stated, the cycloalkenyl group contains 3 to 10 carbon atoms, and additionally, the cycloalkenyl group may be substituted arbitrarily.

The term “alkynyl group” indicates both linear alkynyl radicals and branched alkynyl radicals that have one or more carbon-carbon triple bonds. Unless otherwise stated, the alkynyl group contains 2 to 30 carbon atoms and may include ethynyl, 2-propanyl, and the like, but is not limited thereto. Additionally, the alkynyl group may be substituted arbitrarily.

The term “cycloalkynyl group” used herein indicates cyclic alkynyl radicals. Unless otherwise stated, the cycloalkynyl group contains 3 to 20 carbon atoms, and additionally, the cycloalkynyl group may be substituted arbitrarily.

The terms “aralkyl group” or “arylalkyl group” used herein are used interchangeably and indicates an alkyl group having an aromatic group as a substituent, and additionally, the aralkyl group (arylalkyl group) may be substituted arbitrarily.

The term “aryl group” or “aromatic group” used herein is used with the same meaning, and the aryl group includes both a single monocyclic group and a polycyclic ring group. The polycyclic ring may include a “condensed ring” which are two or more rings where two carbons are shared by two adjacent rings. In addition, the polycyclic ring may also include a form in which two or more rings are simply attached or condensed. Unless otherwise stated, the aryl group contains 6 to 30 carbon atoms and may include phenyl, naphthyl, anthracenyl, phenanthryl, fluorenyl, dimethylfluorenyl, diphenylfluorenyl, spirofluorenyl, and the like, but is not limited thereto. Additionally, the aryl group may be substituted arbitrarily.

the term “heteroaryl group” or “heteroaromatic group” used herein is used with the same meaning, and the heteroaryl group includes both a monocyclic ring group and a polycyclic ring group. The polycyclic ring may include “condensed ring” which are two or more rings where two carbons or hetero elements are shared by two adjacent rings. In addition, the polycyclic ring may also include a form in which two or more rings are simply attached or condensed. Unless otherwise stated, the heteroaryl group contains 1 to 30 carbon atoms, and when the heteroaryl group has 1 or 2 carbon atoms, the heteroaryl group may contain additional hetero elements to form a ring. In addition, the heteroaryl group may contain 1 to 30 carbon atoms, in which one or more carbons in the ring are substituted with a heteroatom, such as oxygen (O), nitrogen (N), sulfur (S), or selenium (Se) and may include 6-membered monocyclic rings, such as pyridyl, pyrazinyl, pyrimidinyl, pyridazinyl, and triazinyl, polycyclic rings, such as phenoxathienyl, indolizinyl, indolyl, purinyl, quinolyl, isoquinolyl, benzooxyzolyl, benzothiazolyl, dibenzooxyzolyl, dibenzothiazolyl, benzoimidazolyl, benzofuranyl, dibenzofuranyl, benzothiophenyl, dibenzothiophenyl, phenylcarbezolyl, 9-phenylcarbazolyl, and carbazolyl, 2-furanyl, N-imidazolyl, 2-isoxazolyl, 2-pyridinyl, 2-pyrimidinyl, and the like, but is not limited thereto. Additionally, the heteroaryl group may be substituted arbitrarily.

The term “heterocyclic ring group” used herein indicates that one or more of the carbon atoms constituting an aryl group, a cycloalkyl group, a cycloalkenyl group, a cycloalkynyl group, an arylalkyl group, an arylamino group, and the like are substituted with a heteroatom, such as oxygen (O), nitrogen (N), or sulfur (S), and with reference to the above definition, includes a heteroaryl group, a heterocycloalkyl group, a heterocycloalkenyl group, a heterocycloalkynyl group, a heteroarylalkyl group, a heteroarylamino group, and the like, and additionally, the hetercyclic group may be substituted arbitrarily.

Unless otherwise stated, the term “carbocyclic ring” used herein may be used as the term including all of “cycloalkyl group”, “cycloalkenyl group”, and “cycloalkynyl group”, which are alicyclic ring groups, and “aryl group (aromatic group)”, which is an aromatic ring group.

The terms “heteroalkyl group”, “heteroalkenyl group”, “heteroalkynyl group”, and “heteroarylalkyl group” used herein indicate that one or more of the carbon atoms constituting the corresponding “alkyl group”, “alkenyl group”, “alkynyl group”, and “aralkyl group (arylalkyl group)” are substituted with heteroatoms, such as oxygen (O), nitrogen (N), and sulfur (S), and additionally, the heteroalkyl group, the heteroalkenyl group, the heteroalkynyl group, and the heteroarylalkyl group may be substituted arbitrarily.

The terms “alkylamino group”, “arylalkylamino group”, “arylamino group”, and “heteroarylamino group” used herein indicate that the alkyl group, the arylalkyl group, the aryl group, and the heteroaryl group are substituted with the amino group (or the amine group) and include all of primary, secondary, and tertiary amino groups (or amine groups), and additionally, the alkylamino group, the arylalkylamino group, the arylamino group, and the heteroarylamino group may be substituted arbitrarily.

The terms “alkylsilyl group”, “arylsilyl group”, “alkoxy group”, “aryloxy group”, “alkylthio group”, and “arylthio group” indicate that each of the alkyl group and the aryl group is substituted with the silyl group, the oxy group, or the thio group, and additionally, the alkylsilyl group, the arylsilyl group, the alkoxy group, the aryloxy group, the alkylthio group, and the arylthio group may be substituted arbitrarily.

The terms “arylene group”, “arylalkylene group”, “heteroarylene group”, and “heteroarylalkylene group” indicate that each of the aryl group, the arylalkyl group, the heteroaryl group, and the heteroarylalkyl group has a divalent substituent further including one substitution. Additionally, the arylene group, the arylalkylene group, the heteroarylene group, and the heteroarylalkylene group may be substituted arbitrarily.

The term “substituted” indicates that, instead of a hydrogen atom (H) being bonded to a carbon atom, another substituent is bonded to the corresponding carbon atom. A substituent for the case of being “substituted” may be with a single substituent or a plurality of substituents. When a plurality of substituents are present, and each substituent may be the same as or different from each other.

The substituent may be selected from the group consisting of deuterium; a cyano group; a nitro group; a halogen group; a hydroxy group; an alkyl group having 1 to 30 carbon atoms; an alkenyl group having 2 to 30 carbon atoms; an alkynyl group having 2 to 24 carbon atoms; a heteroalkyl group having 2 to 30 carbon atoms; an aralkyl group having 6 to 30 carbon atoms; a cycloalkyl group having 3 to 20 carbon atoms; a heterocycloalkyl group having 3 to 20 carbon atoms; an aryl group having 6 to 30 carbon atoms; a heteroaryl group having 2 to 30 carbon atoms; a heteroarylalkyl group having 3 to 30 carbon atoms; an alkoxy group having 1 to 30 carbon atoms; an alkylsilyl group having 1 to 30 carbon atoms; an arylsilyl group having 6 to 30 carbon atoms; and an aryloxy group having 6 to 30 carbon atoms.

Unless otherwise stated herein, a position to be substituted is not limited as long as it is a position where a hydrogen atom is substituted, that is, a position where a substituent may be substituted, and when two or more substituents are present, a substituent is the same as or different from each other.

The objects and substituents as defined herein may be the same as or different from each other unless otherwise stated.

In the present specification, the standard for units is based on weight (wt) unless specifically stated. For example, when “%” is described, it is construed as weight % (wt %).

Hereinafter, an organic compound and an organic light emitting diode including the same according to the present disclosure will be described in detail.

The organic compound according to the present disclosure may be represented by Chemical Formula 1 below:

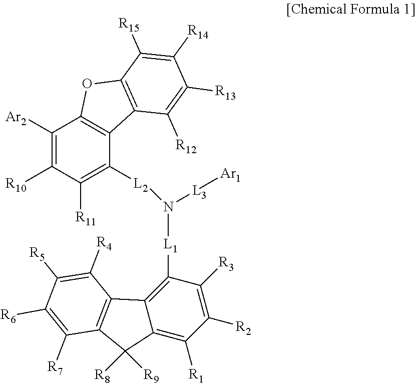

in Chemical Formula 1,

L₁ to L₃ are, each independently, selected from the group consisting of a single bond; a substituted or unsubstituted aryl group having 6 to 30 carbon atoms; a substituted or unsubstituted aralkyl group having 7 to 30 carbon atoms; a substituted or unsubstituted heteroaryl group having 5 to 60 carbon atoms; and a substituted or unsubstituted heteroarylalkyl group having 6 to 60 carbon atoms,

R₁ to R₁₅, Ar₁ and Ar₂ are, each independently, selected from the group consisting of hydrogen; deuterium; a cyano group; a nitro group; a halogen group; a hydroxy group; a substituted or unsubstituted alkyl group having 1 to 30 carbon atoms; a substituted or unsubstituted alkenyl group having 2 to 30 carbon atoms; a substituted or unsubstituted alkynyl group having 2 to 24 carbon atoms; a substituted or unsubstituted heteroalkyl group having 2 to 30 carbon atoms; a substituted or unsubstituted aryl group having 6 to 30 carbon atoms; a substituted or unsubstituted heteroaryl group having 2 to 30 carbon atoms; a substituted or unsubstituted heteroarylalkyl group having 3 to 30 carbon atoms; a substituted or unsubstituted aralkyl group having 6 to 30 carbon atoms; a substituted or unsubstituted cycloalkyl group having 3 to 20 carbon atoms; a substituted or unsubstituted heterocycloalkyl group having 3 to 20 carbon atoms; a substituted or unsubstituted alkoxy group having 1 to 30 carbon atoms; a substituted or unsubstituted alkylsilyl group having 1 to 30 carbon atoms; a substituted or unsubstituted arylsilyl group having 6 to 30 carbon atoms; and a substituted or unsubstituted aryloxy group having 6 to 30 carbon atoms, and

optionally, when L₁ to L₃, R₁ to R₁₅, and Ar₁ and Ar₂ are substituted, substituents may be one or more selected from the group consisting of deuterium; a cyano group; a nitro group; halogen; a hydroxy group; an alkyl group having 1 to 30 carbon atoms; an alkenyl group having 2 to 30 carbon atoms; an alkynyl group having 2 to 24 carbon atoms; a heteroalkyl group having 2 to 30 carbon atoms; an aralkyl group having 6 to 30 carbon atoms; a cycloalkyl group having 3 to 20 carbon atoms; a heterocycloalkyl group having 3 to 20 carbon atoms; an aryl group having 6 to 30 carbon atoms; a heteroaryl group having 2 to 30 carbon atoms; a heteroarylalkyl group having 3 to 30 carbon atoms; an alkoxy group having 1 to 30 carbon atoms; an alkylsilyl group having 1 to 30 carbon atoms; an arylsilyl group having 6 to 30 carbon atoms; and an aryloxy group having 6 to 30 carbon atoms; and when a plurality of substituents are present, each substituent may be the same as or different from each other.

A fourth position of the dimethylfluorene group and a first position of the dibenzofuran group in the compound represented by Chemical Formula 1 may be bonded to the nitrogen (N) of the amine group through linkers L₁ and L₂, respectively. Additionally, the substituent Ar₂ may be bonded to the fourth position of the dibenzofuran group to increase conjugation and expand the electron cloud of HOMO, thereby improving hole injection and hole transport characteristics. In addition, the compound represented by Chemical Formula 1 may have a suitable energy level as a hole transport auxiliary layer that serves to transfer holes from the hole transport layer to the emitting layer and block electrons coming from the emitting layer, and thus provide the characteristics suitable as a material for the hole transport auxiliary layer.

According to one embodiment of the present disclosure, L₁ and L₂ may each be selected as a single bond.

According to one embodiment of the present disclosure, L₃ may be selected from the group consisting of a single bond; a substituted or unsubstituted aryl group having 6 to 30 carbon atoms; and a substituted or unsubstituted heteroaryl group having 2 to 30 carbon atoms.

According to one embodiment of the present disclosure, Ar₁ may be selected from the group consisting of a substituted or unsubstituted aryl group having 6 to 30 carbon atoms; a substituted or unsubstituted heteroaryl group having 2 to 30 carbon atoms; and a substituted or unsubstituted aralkyl group having 6 to 30 carbon atoms. For example, Ar₁ may be one of a substituted or unsubstituted phenyl group, a substituted or unsubstituted naphthyl group, a substituted or unsubstituted biphenyl group, a substituted or unsubstituted phenanthrene group, a substituted or unsubstituted dimethyl fluorene group, a substituted or unsubstituted dibenzofuran group, a substituted or unsubstituted dibenzothiophene group, and a substituted or unsubstituted carbazole group.

According to one embodiment of the present disclosure, Ar₂ may be a substituted or unsubstituted aryl group having 6 to 30 carbon atoms. For example, Ar₂ may be one of a substituted or unsubstituted phenyl group, a substituted or unsubstituted naphthyl group, and a substituted or unsubstituted phenanthrene group.

According to one embodiment of the present disclosure, Chemical Formula 1 may be one selected from the group consisting of Chemical Formulas 2 to 5 below:

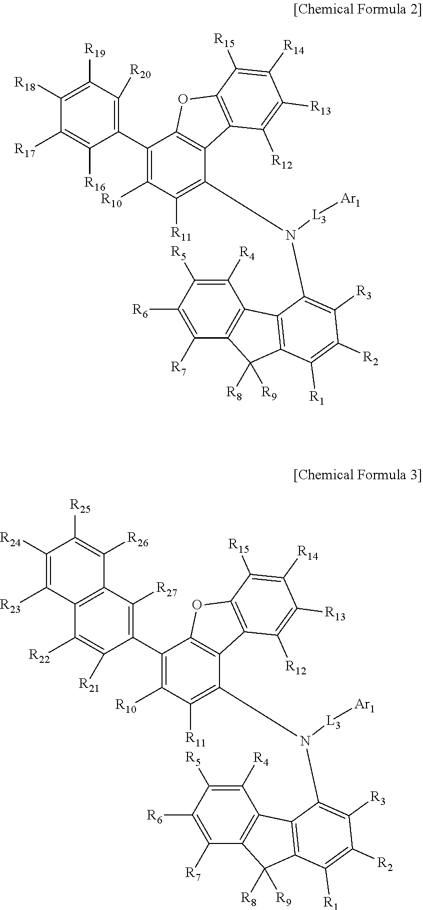

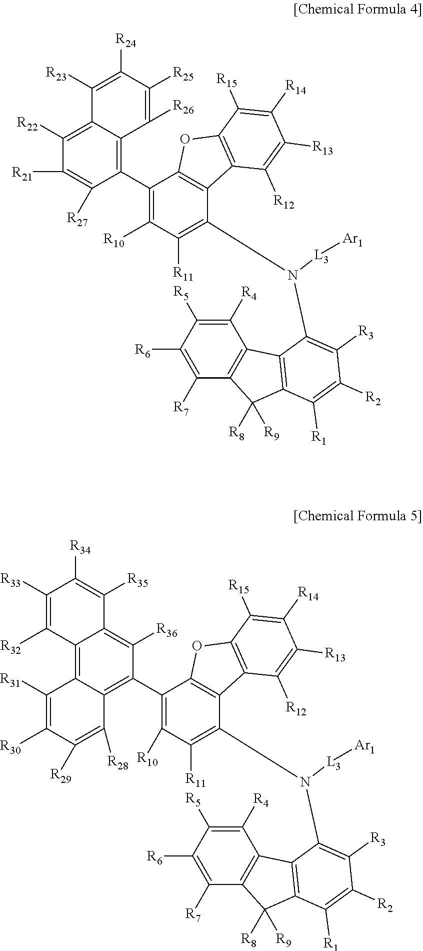

in Chemical Formulas 2 to 5,

R₁ to R₁₅, L₃, and Ar₁ are as defined in Chemical Formula 1,

R₁₆ to R₃₆ may, each independently, be one selected from the group consisting of hydrogen; deuterium; a cyano group; a nitro group; halogen; a hydroxy group; a substituted or unsubstituted alkyl group having 1 to 30 carbon atoms; a substituted or unsubstituted alkenyl group having 2 to 30 carbon atoms; a substituted or unsubstituted alkynyl group having 2 to 24 carbon atoms; a substituted or unsubstituted heteroalkyl group having 2 to 30 carbon atoms; a substituted or unsubstituted aryl group having 6 to 30 carbon atoms; a substituted or unsubstituted heteroaryl group having 2 to 30 carbon atoms; a substituted or unsubstituted heteroarylalkyl group having 3 to 30 carbon atoms; a substituted or unsubstituted aralkyl group having 6 to 30 carbon atoms; a substituted or unsubstituted cycloalkyl group having 3 to 20 carbon atoms; a substituted or unsubstituted heterocycloalkyl group having 3 to 20 carbon atoms; a substituted or unsubstituted alkoxy group having 1 to 30 carbon atoms; a substituted or unsubstituted alkylsilyl group having 1 to 30 carbon atoms; a substituted or unsubstituted arylsilyl group having 6 to 30 carbon atoms; and a substituted or unsubstituted aryloxy group having 6 to 30 carbon atoms, and

optionally, when R₁₆ to R₃₆ are substituted, substituents may be one or more selected from the group consisting of deuterium; a cyano group; a nitro group; halogen; a hydroxy group; an alkyl group having 1 to 30 carbon atoms; an alkenyl group having 2 to 30 carbon atoms; an alkynyl group having 2 to 24 carbon atoms; a heteroalkyl group having 2 to 30 carbon atoms; an aralkyl group having 6 to 30 carbon atoms; a cycloalkyl group having 3 to 20 carbon atoms; a heterocycloalkyl group having 3 to 20 carbon atoms; an aryl group having 6 to 30 carbon atoms; a heteroaryl group having 2 to 30 carbon atoms; a heteroarylalkyl group having 3 to 30 carbon atoms; an alkoxy group having 1 to 30 carbon atoms; an alkylsilyl group having 1 to 30 carbon atoms; an arylsilyl group having 6 to 30 carbon atoms; and an aryloxy group having 6 to 30 carbon atoms, and when a plurality of substituents are present, each substituent may be the same as or different from each other.

According to one embodiment of the present disclosure, Chemical Formula 1 may be one selected from the group consisting of Chemical Formulas 6 to 9 below:

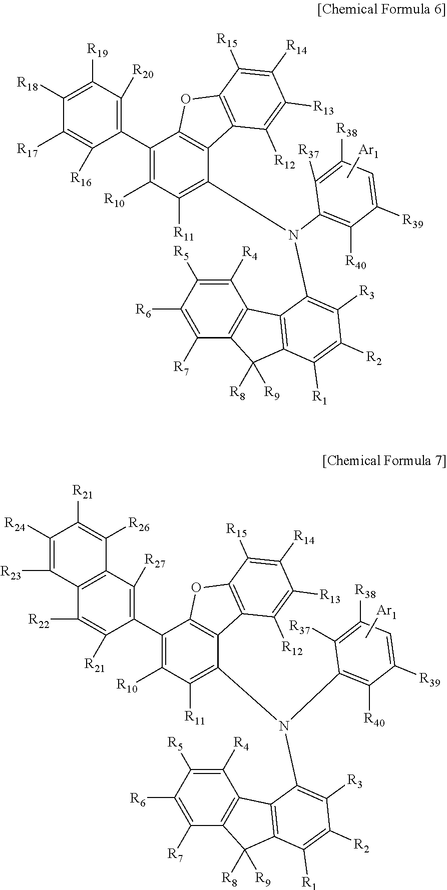

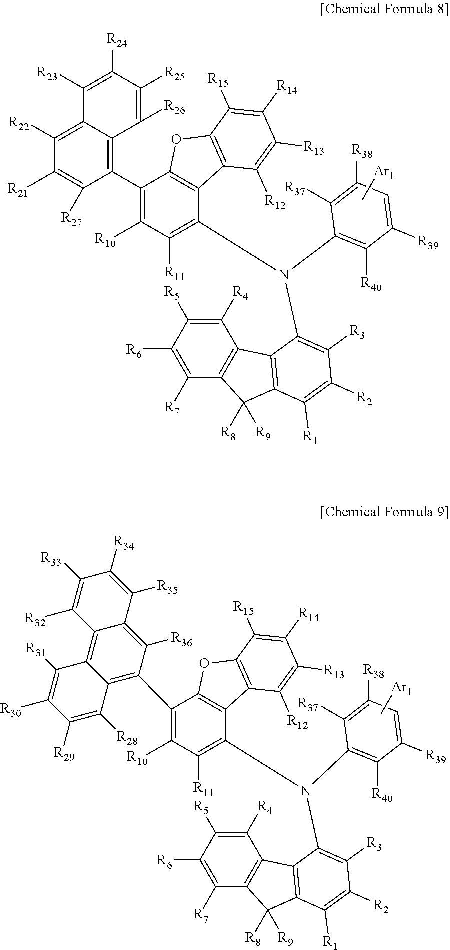

in Chemical Formulas 6 to 9,

R₁ to R₁₅ and Ar₁ are as defined in Chemical Formula 1,

R₁₆ to R₄₀ may, each independently, be one selected from the group consisting of hydrogen; deuterium; a cyano group; a nitro group; halogen; a hydroxy group; a substituted or unsubstituted alkyl group having 1 to 30 carbon atoms; a substituted or unsubstituted alkenyl group having 2 to 30 carbon atoms; a substituted or unsubstituted alkynyl group having 2 to 24 carbon atoms; a substituted or unsubstituted heteroalkyl group having 2 to 30 carbon atoms; a substituted or unsubstituted aryl group having 6 to 30 carbon atoms; a substituted or unsubstituted heteroaryl group having 2 to 30 carbon atoms; a substituted or unsubstituted heteroarylalkyl group having 3 to 30 carbon atoms; a substituted or unsubstituted aralkyl group having 6 to 30 carbon atoms; a substituted or unsubstituted cycloalkyl group having 3 to 20 carbon atoms; a substituted or unsubstituted heterocycloalkyl group having 3 to 20 carbon atoms; a substituted or unsubstituted alkoxy group having 1 to 30 carbon atoms; a substituted or unsubstituted alkylsilyl group having 1 to 30 carbon atoms; a substituted or unsubstituted arylsilyl group having 6 to 30 carbon atoms; and a substituted or unsubstituted aryloxy group having 6 to 30 carbon atoms, and

optionally, when R₁₆ to R₄₀ are substituted, substituents may be one or more selected from the group consisting of deuterium; a cyano group; a nitro group; halogen; a hydroxy group; an alkyl group having 1 to 30 carbon atoms; an alkenyl group having 2 to 30 carbon atoms; an alkynyl group having 2 to 24 carbon atoms; a heteroalkyl group having 2 to 30 carbon atoms; an aralkyl group having 6 to 30 carbon atoms; a cycloalkyl group having 3 to 20 carbon atoms; a heterocycloalkyl group having 3 to 20 carbon atoms; an aryl group having 6 to 30 carbon atoms; a heteroaryl group having 2 to 30 carbon atoms; a heteroarylalkyl group having 3 to 30 carbon atoms; an alkoxy group having 1 to 30 carbon atoms; an alkylsilyl group having 1 to 30 carbon atoms; an arylsilyl group having 6 to 30 carbon atoms; and an aryloxy group having 6 to 30 carbon atoms, and when a plurality of substituents are present, each substituent may be the same as or different from each other.

According to one embodiment of the present disclosure, when a substituent is present in R₁ to R₄₀, L₁ to L₃, Ar₁ and Ar₂, the substituent may, each independently, be selected from deuterium, an alkyl group having 1 to 30 carbon atoms, an aryl group having 6 to 30 carbon atoms, a heteroaryl group having 5 to 30 carbon atoms, and a heteroarylalkyl group having 3 to 30 carbon atoms.

According to one embodiment of the present disclosure, the compound represented by Chemical Formula 1 may be any one selected from the following compounds, and the following compounds may be additionally substituted.

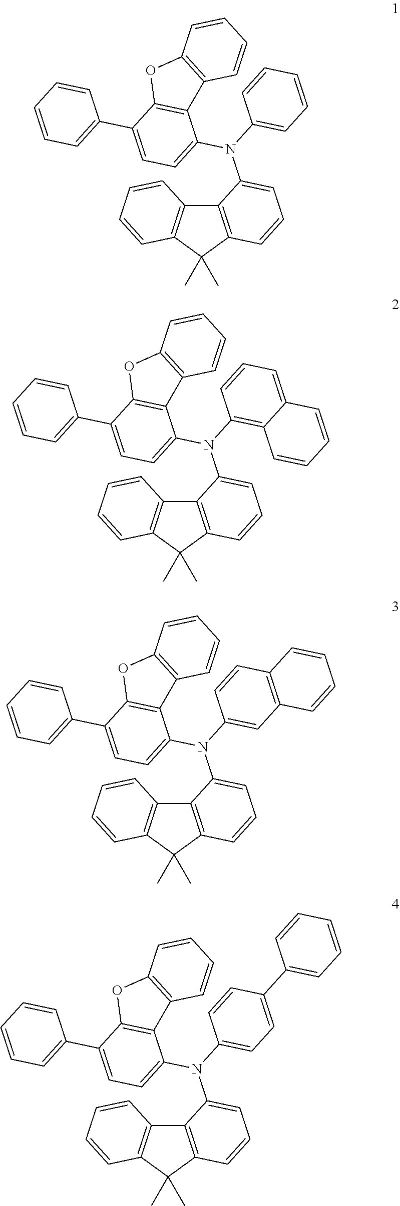

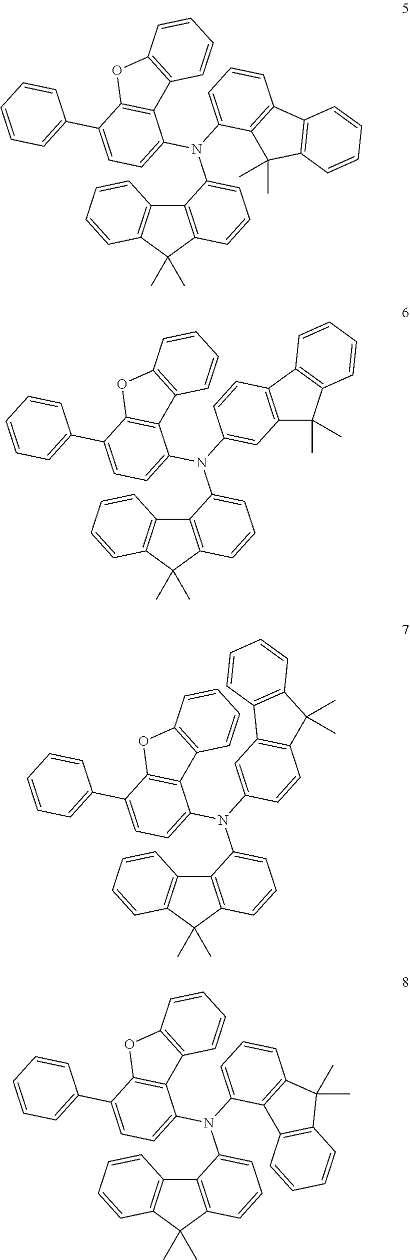

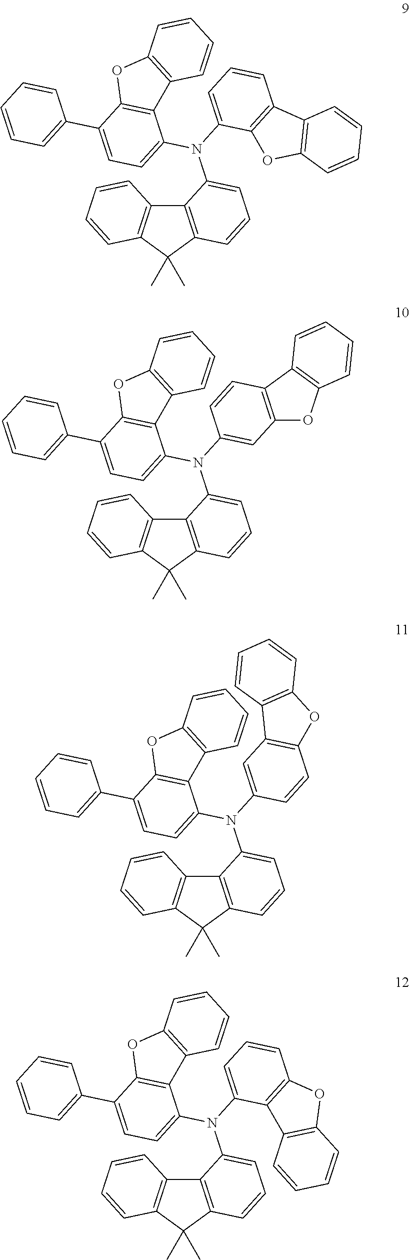

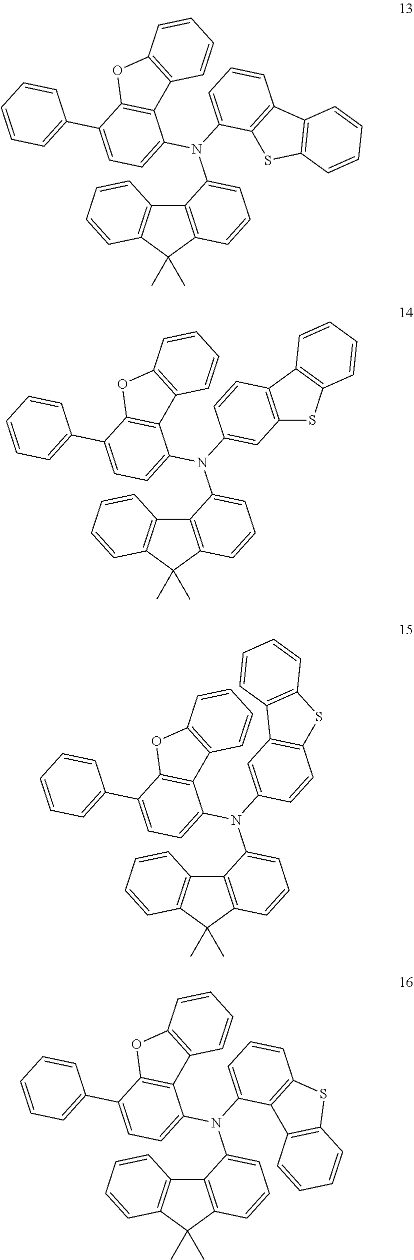

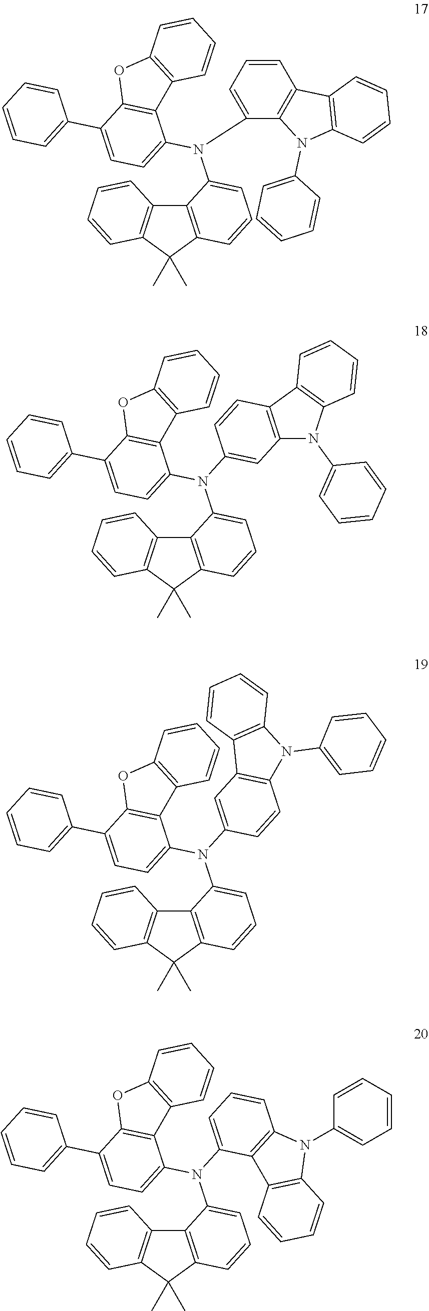 [SMILES: CC1(C)c2ccccc2-c2c(N(c3ccc(-c4ccccc4)c4oc5ccccc5c34)c3cccc4c5ccccc5n(-c5ccccc5)c34)cccc21 ; CC1(C)c2ccccc2-c2c(N(c3ccc4c5ccccc5n(-c5ccccc5)c4c3)c3ccc(-c4ccccc4)c4oc5ccccc5c34)cccc21 ; CC1(C)c2ccccc2-c2c(N(c3ccc4c(c3)c3ccccc3n4-c3ccccc3)c3ccc(-c4ccccc4)c4oc5ccccc5c34)cccc21 ; CC1(C)c2ccccc2-c2c(N(c3ccc(-c4ccccc4)c4oc5ccccc5c34)c3cccc4c3c3ccccc3n4-c3ccccc3)cccc21]

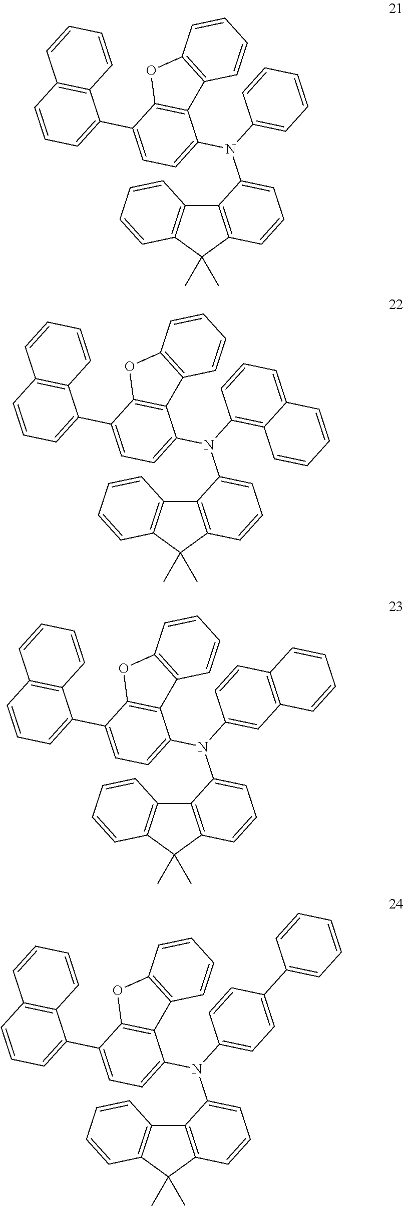

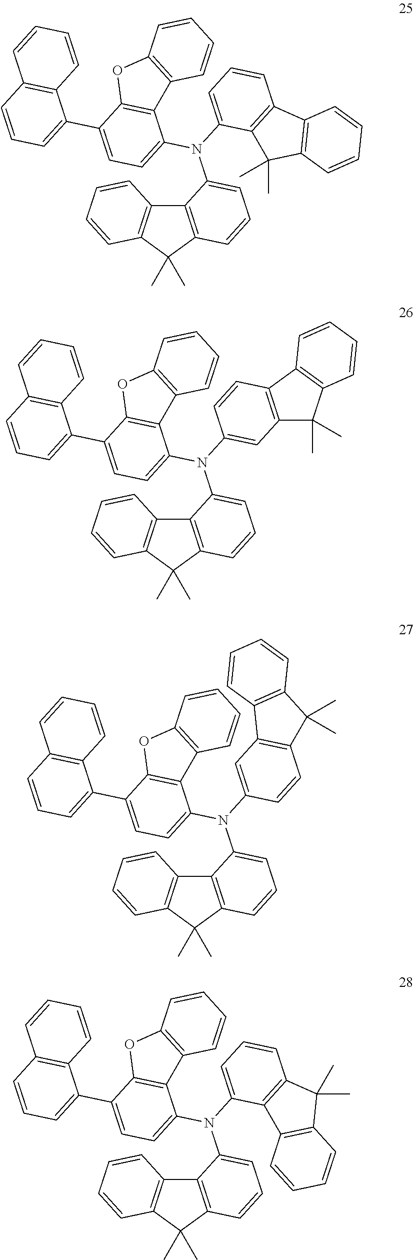

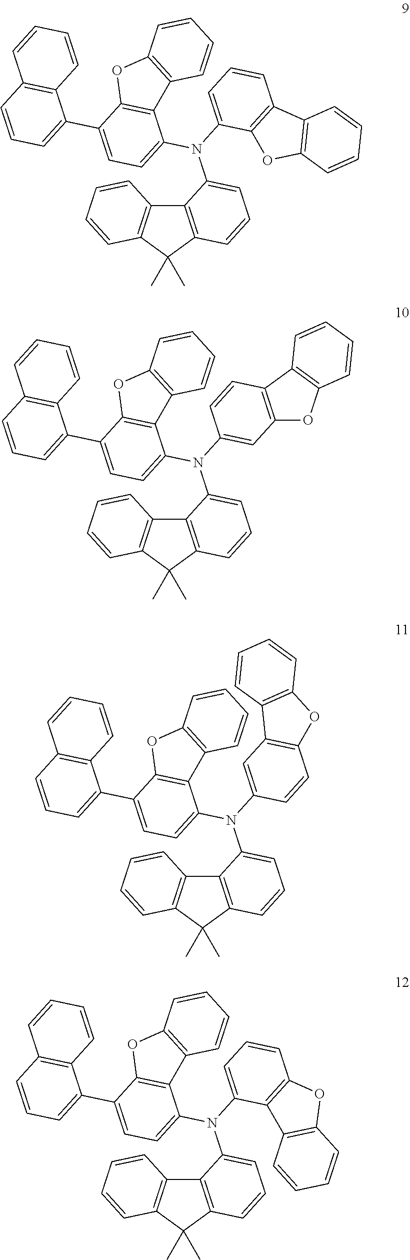

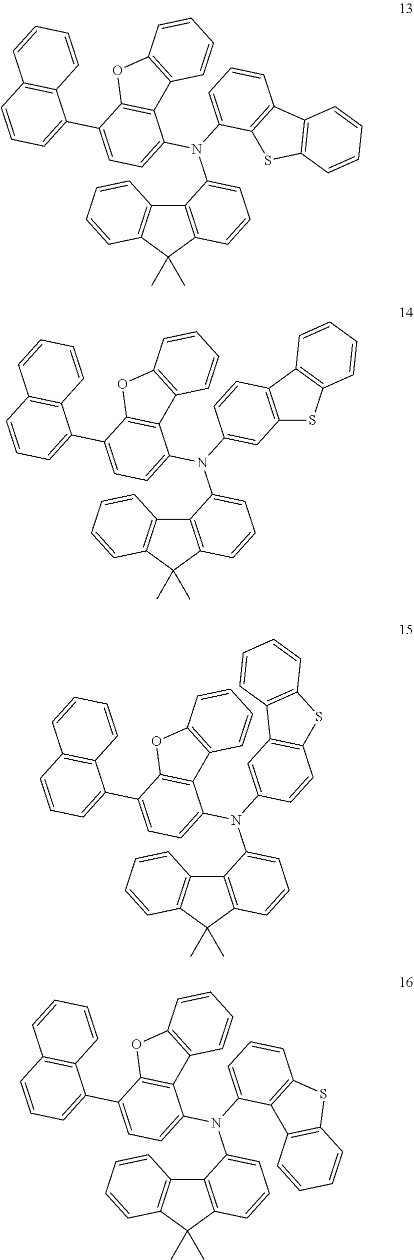

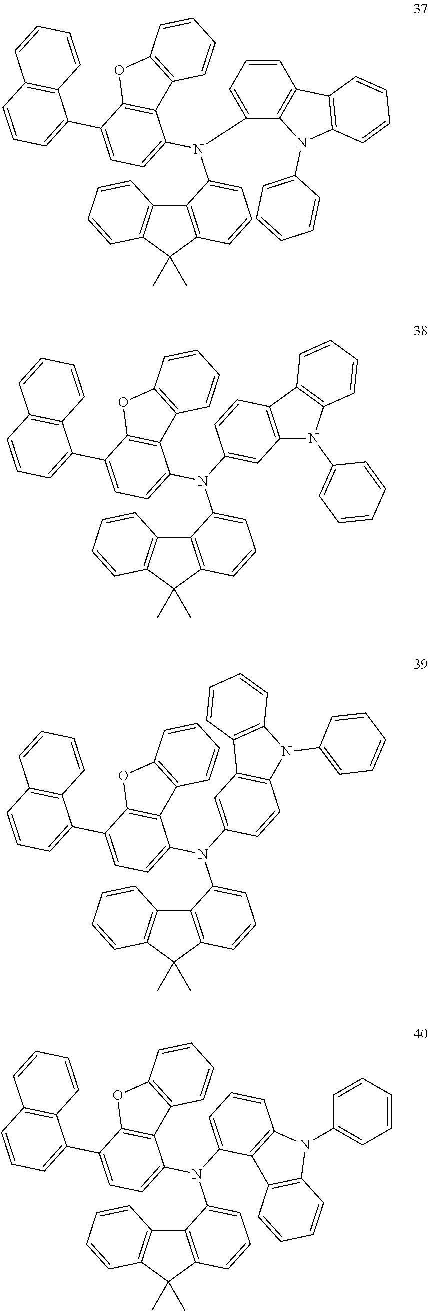 [SMILES: CC1(C)c2ccccc2-c2c(N(c3ccc(-c4cccc5ccccc45)c4oc5ccccc5c34)c3cccc4c5ccccc5n(-c5ccccc5)c34)cccc21 ; CC1(C)c2ccccc2-c2c(N(c3ccc4c5ccccc5n(-c5ccccc5)c4c3)c3ccc(-c4cccc5ccccc45)c4oc5ccccc5c34)cccc21 ; CC1(C)c2ccccc2-c2c(N(c3ccc4c(c3)c3ccccc3n4-c3ccccc3)c3ccc(-c4cccc5ccccc45)c4oc5ccccc5c34)cccc21 ; CC1(C)c2ccccc2-c2c(N(c3ccc(-c4cccc5ccccc45)c4oc5ccccc5c34)c3cccc4c3c3ccccc3n4-c3ccccc3)cccc21]

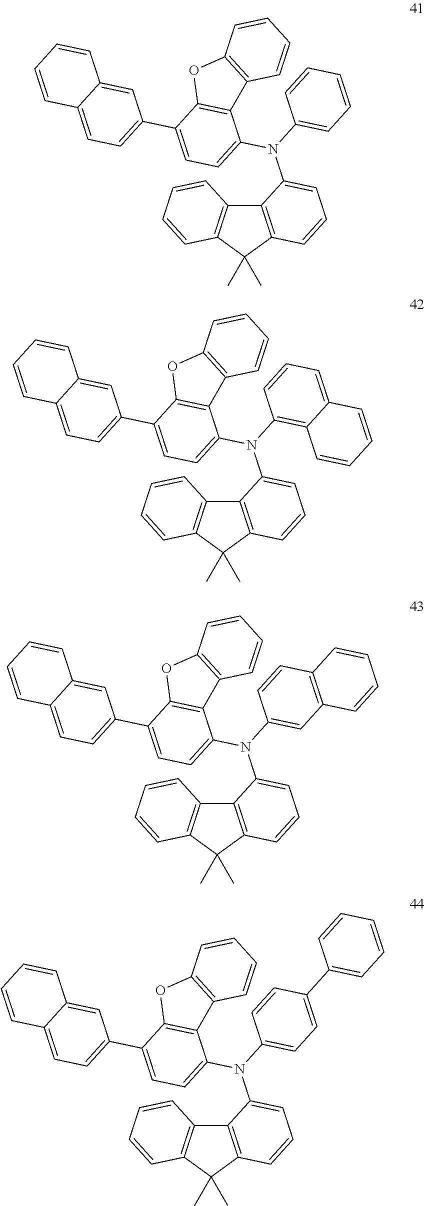

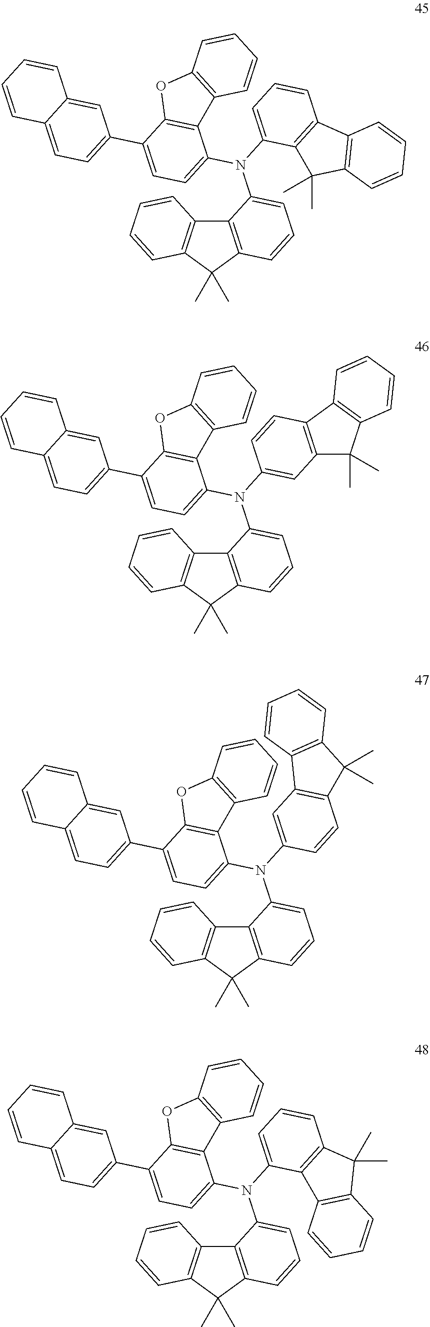 [SMILES: CC1(C)c2ccccc2-c2c(N(c3cccc4c3C(C)(C)c3ccccc3-4)c3ccc(-c4ccc5ccccc5c4)c4oc5ccccc5c34)cccc21 ; CC1(C)c2ccccc2-c2ccc(N(c3cccc4c3-c3ccccc3C4(C)C)c3ccc(-c4ccc5ccccc5c4)c4oc5ccccc5c34)cc21 ; CC1(C)c2ccccc2-c2cc(N(c3cccc4c3-c3ccccc3C4(C)C)c3ccc(-c4ccc5ccccc5c4)c4oc5ccccc5c34)ccc21 ; CC1(C)c2ccccc2-c2c(N(c3cccc4c3-c3ccccc3C4(C)C)c3ccc(-c4ccc5ccccc5c4)c4oc5ccccc5c34)cccc21]

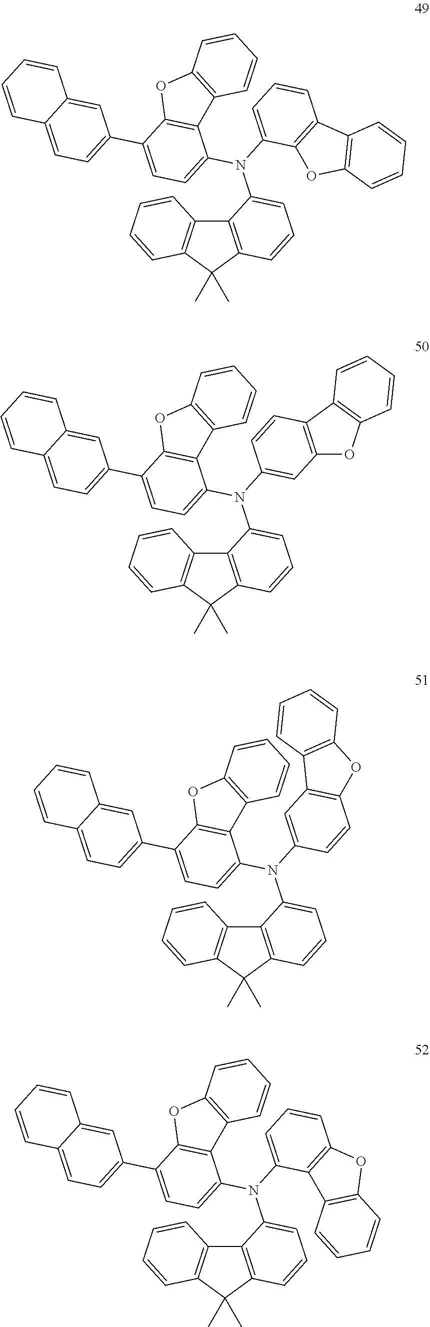 [SMILES: CC1(C)c2ccccc2-c2c(N(c3cccc4c3oc3ccccc34)c3ccc(-c4ccc5ccccc5c4)c4oc5ccccc5c34)cccc21 ; CC1(C)c2ccccc2-c2c(N(c3ccc4c(c3)oc3ccccc34)c3ccc(-c4ccc5ccccc5c4)c4oc5ccccc5c34)cccc21 ; CC1(C)c2ccccc2-c2c(N(c3ccc4oc5ccccc5c4c3)c3ccc(-c4ccc5ccccc5c4)c4oc5ccccc5c34)cccc21 ; CC1(C)c2ccccc2-c2c(N(c3cccc4oc5ccccc5c34)c3ccc(-c4ccc5ccccc5c4)c4oc5ccccc5c34)cccc21]

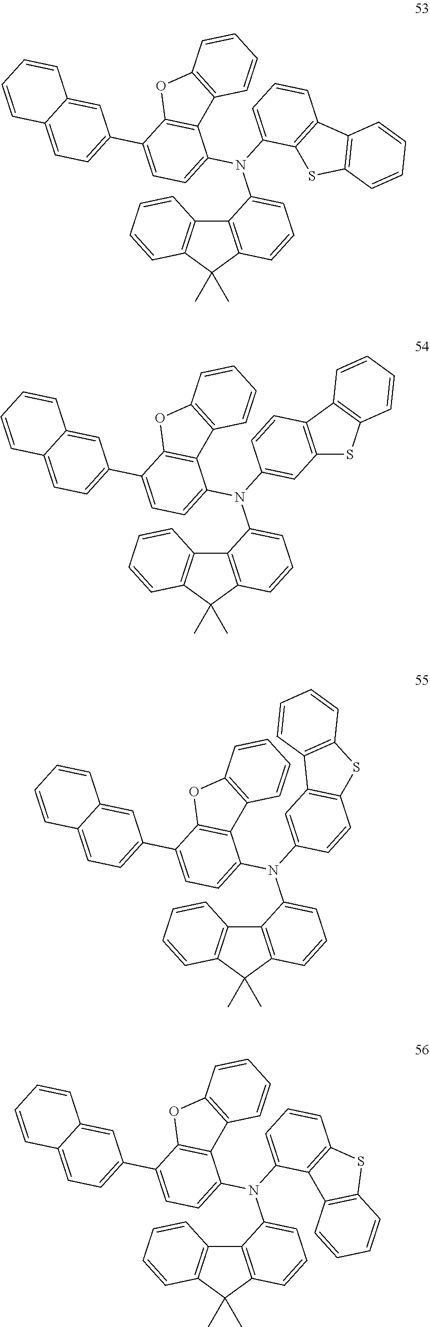 [SMILES: CC1(C)c2ccccc2-c2c(N(c3cccc4c3sc3ccccc34)c3ccc(-c4ccc5ccccc5c4)c4oc5ccccc5c34)cccc21 ; CC1(C)c2ccccc2-c2c(N(c3ccc4c(c3)sc3ccccc34)c3ccc(-c4ccc5ccccc5c4)c4oc5ccccc5c34)cccc21 ; CC1(C)c2ccccc2-c2c(N(c3ccc4sc5ccccc5c4c3)c3ccc(-c4ccc5ccccc5c4)c4oc5ccccc5c34)cccc21 ; CC1(C)c2ccccc2-c2c(N(c3cccc4sc5ccccc5c34)c3ccc(-c4ccc5ccccc5c4)c4oc5ccccc5c34)cccc21]

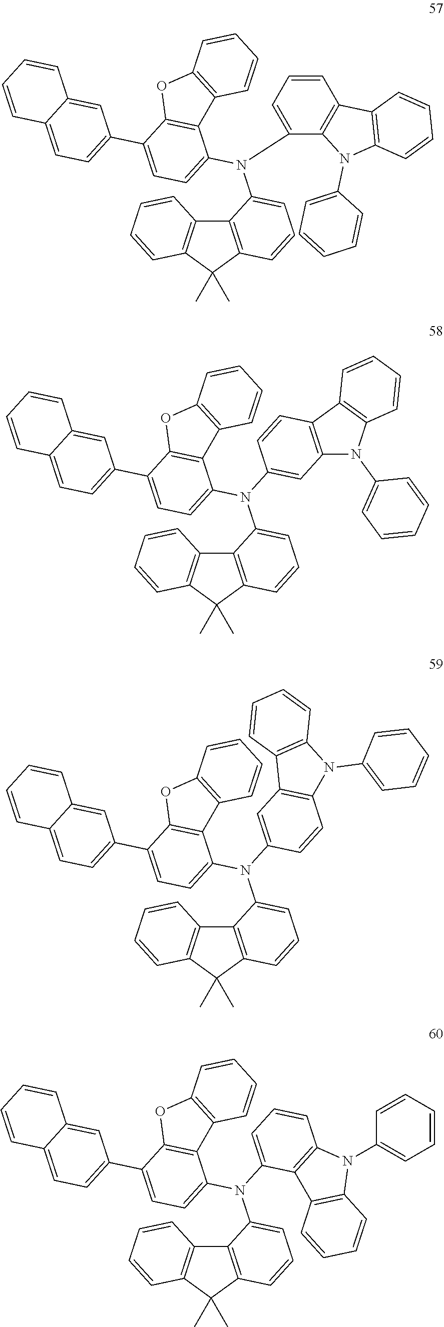 [SMILES: CC1(C)c2ccccc2-c2c(N(c3ccc(-c4ccc5ccccc5c4)c4oc5ccccc5c34)c3cccc4c5ccccc5n(-c5ccccc5)c34)cccc21 ; CC1(C)c2ccccc2-c2c(N(c3ccc4c5ccccc5n(-c5ccccc5)c4c3)c3ccc(-c4ccc5ccccc5c4)c4oc5ccccc5c34)cccc21 ; CC1(C)c2ccccc2-c2c(N(c3ccc4c(c3)c3ccccc3n4-c3ccccc3)c3ccc(-c4ccc5ccccc5c4)c4oc5ccccc5c34)cccc21 ; CC1(C)c2ccccc2-c2c(N(c3ccc(-c4ccc5ccccc5c4)c4oc5ccccc5c34)c3cccc4c3c3ccccc3n4-c3ccccc3)cccc21]

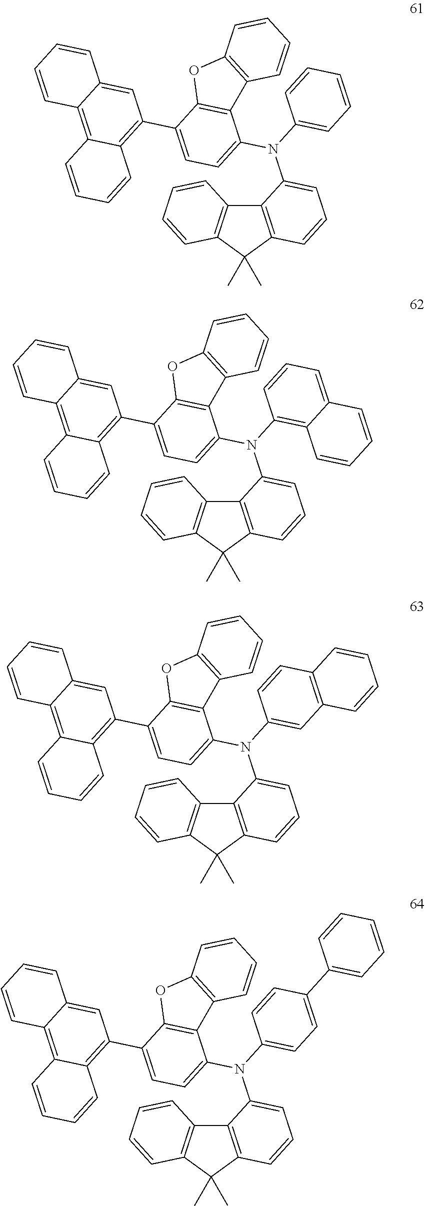

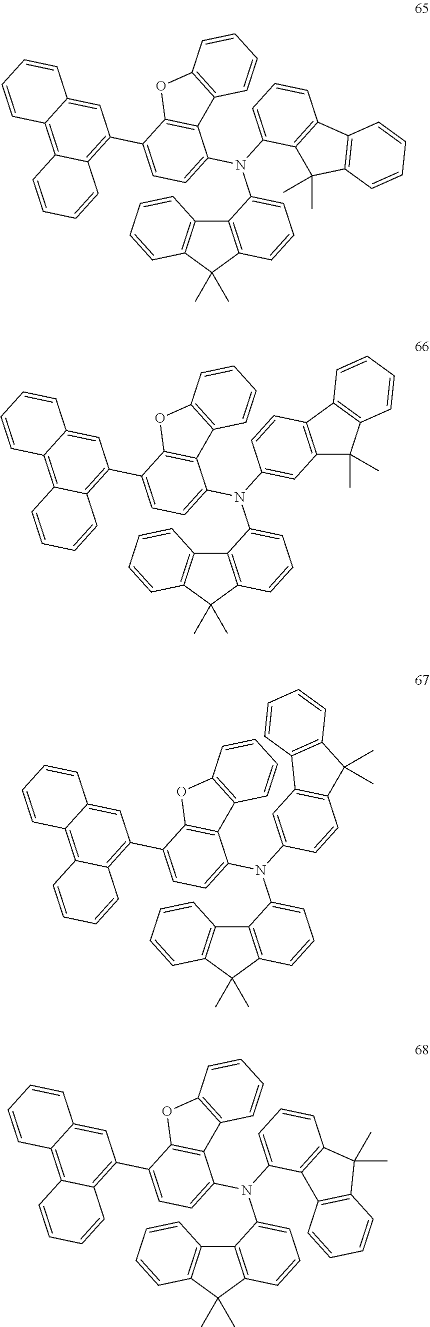 [SMILES: CC1(C)c2ccccc2-c2c(N(c3cccc4c3C(C)(C)c3ccccc3-4)c3ccc(-c4cc5ccccc5c5ccccc45)c4oc5ccccc5c34)cccc21 ; CC1(C)c2ccccc2-c2ccc(N(c3cccc4c3-c3ccccc3C4(C)C)c3ccc(-c4cc5ccccc5c5ccccc45)c4oc5ccccc5c34)cc21 ; CC1(C)c2ccccc2-c2cc(N(c3cccc4c3-c3ccccc3C4(C)C)c3ccc(-c4cc5ccccc5c5ccccc45)c4oc5ccccc5c34)ccc21 ; CC1(C)c2ccccc2-c2c(N(c3cccc4c3-c3ccccc3C4(C)C)c3ccc(-c4cc5ccccc5c5ccccc45)c4oc5ccccc5c34)cccc21]

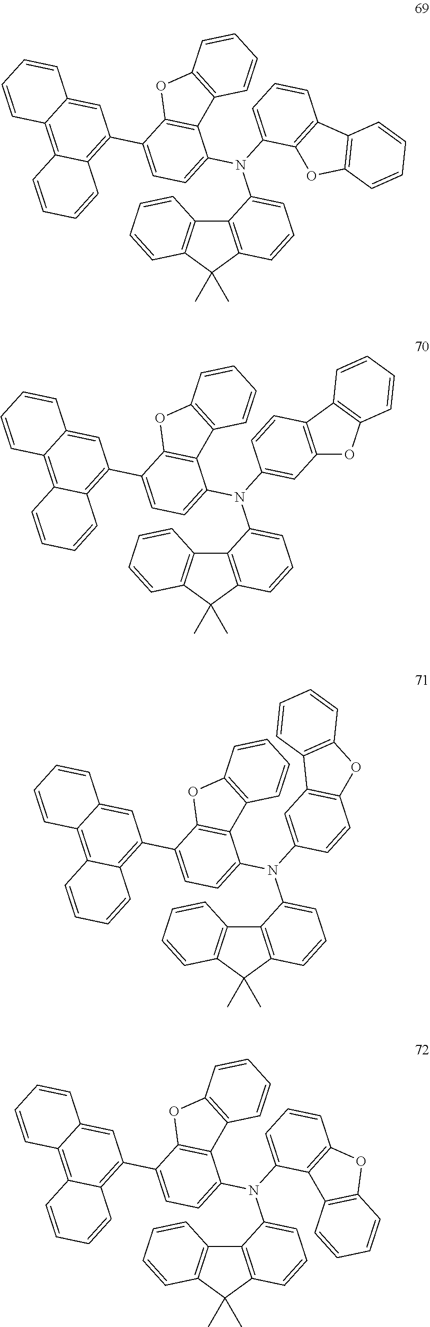 [SMILES: CC1(C)c2ccccc2-c2c(N(c3cccc4c3oc3ccccc34)c3ccc(-c4cc5ccccc5c5ccccc45)c4oc5ccccc5c34)cccc21 ; CC1(C)c2ccccc2-c2c(N(c3ccc4c(c3)oc3ccccc34)c3ccc(-c4cc5ccccc5c5ccccc45)c4oc5ccccc5c34)cccc21 ; CC1(C)c2ccccc2-c2c(N(c3ccc4oc5ccccc5c4c3)c3ccc(-c4cc5ccccc5c5ccccc45)c4oc5ccccc5c34)cccc21 ; CC1(C)c2ccccc2-c2c(N(c3cccc4oc5ccccc5c34)c3ccc(-c4cc5ccccc5c5ccccc45)c4oc5ccccc5c34)cccc21]

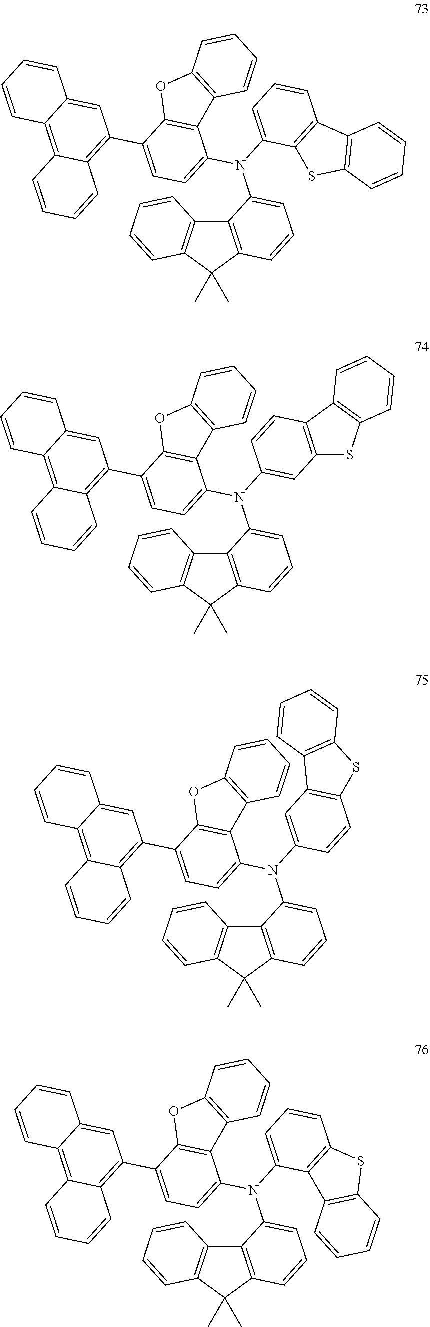 [SMILES: CC1(C)c2ccccc2-c2c(N(c3cccc4c3sc3ccccc34)c3ccc(-c4cc5ccccc5c5ccccc45)c4oc5ccccc5c34)cccc21 ; CC1(C)c2ccccc2-c2c(N(c3ccc4c(c3)sc3ccccc34)c3ccc(-c4cc5ccccc5c5ccccc45)c4oc5ccccc5c34)cccc21 ; CC1(C)c2ccccc2-c2c(N(c3ccc4sc5ccccc5c4c3)c3ccc(-c4cc5ccccc5c5ccccc45)c4oc5ccccc5c34)cccc21 ; CC1(C)c2ccccc2-c2c(N(c3cccc4sc5ccccc5c34)c3ccc(-c4cc5ccccc5c5ccccc45)c4oc5ccccc5c34)cccc21]

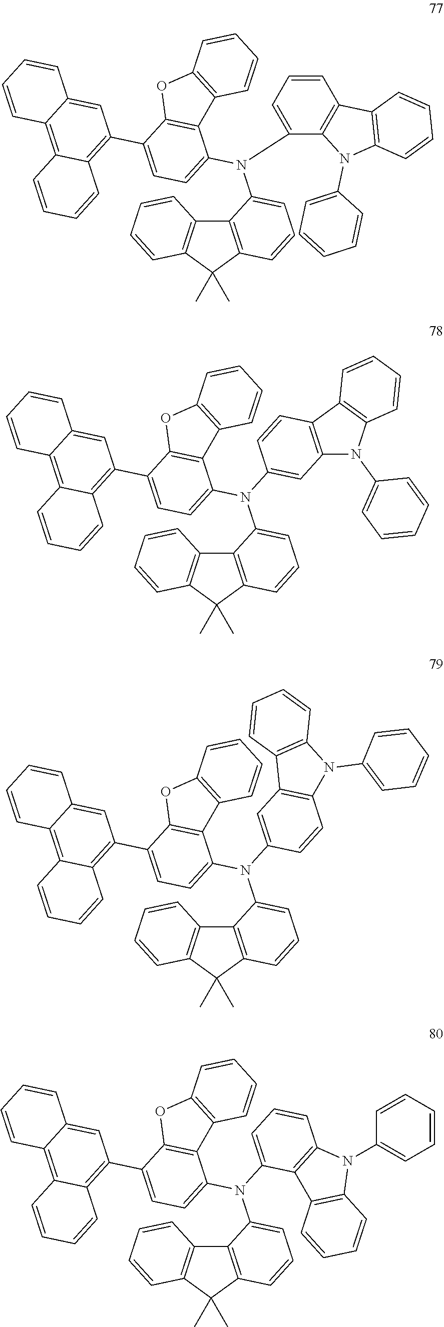 [SMILES: CC1(C)c2ccccc2-c2c(N(c3ccc(-c4cc5ccccc5c5ccccc45)c4oc5ccccc5c34)c3cccc4c5ccccc5n(-c5ccccc5)c34)cccc21 ; CC1(C)c2ccccc2-c2c(N(c3ccc4c5ccccc5n(-c5ccccc5)c4c3)c3ccc(-c4cc5ccccc5c5ccccc45)c4oc5ccccc5c34)cccc21 ; CC1(C)c2ccccc2-c2c(N(c3ccc4c(c3)c3ccccc3n4-c3ccccc3)c3ccc(-c4cc5ccccc5c5ccccc45)c4oc5ccccc5c34)cccc21 ; CC1(C)c2ccccc2-c2c(N(c3ccc(-c4cc5ccccc5c5ccccc45)c4oc5ccccc5c34)c3cccc4c3c3ccccc3n4-c3ccccc3)cccc21]

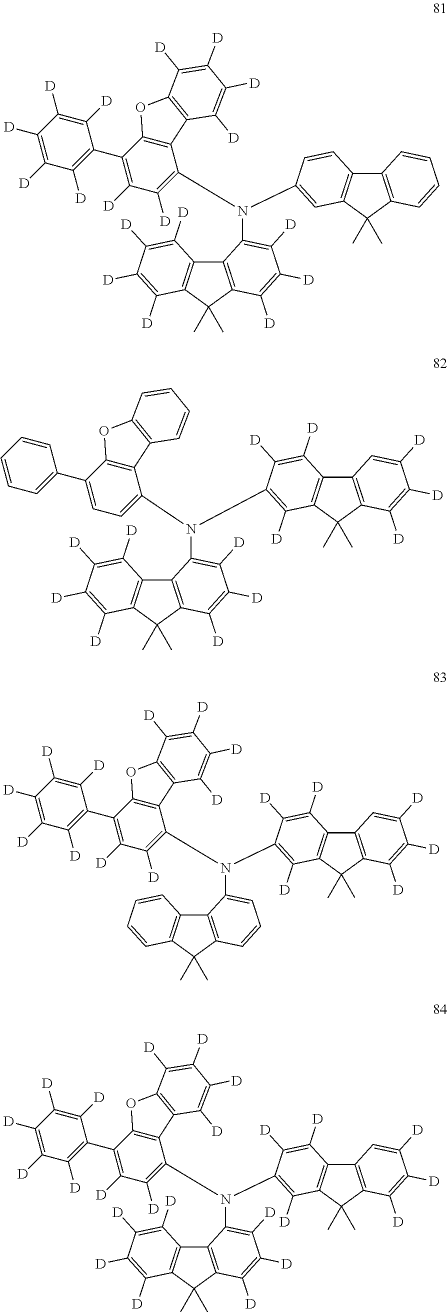

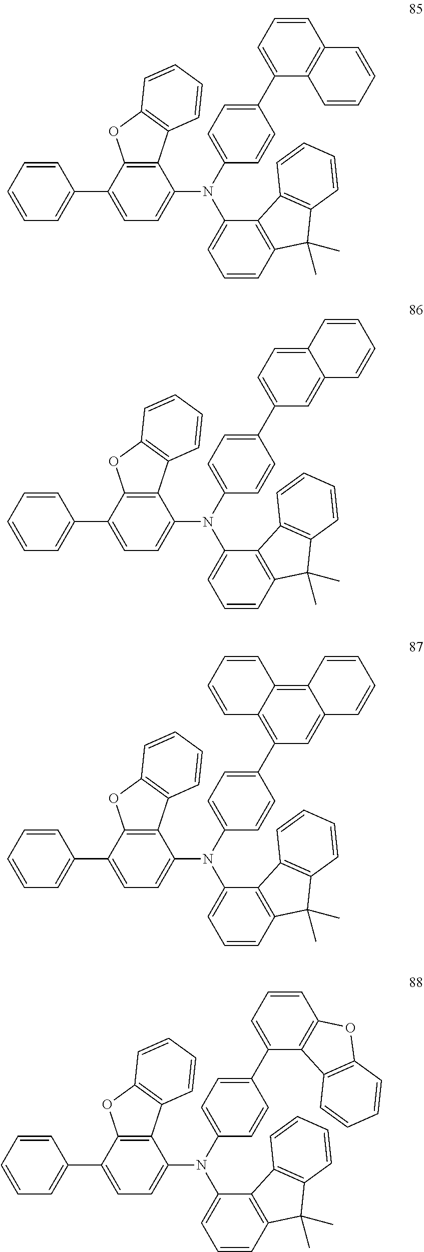

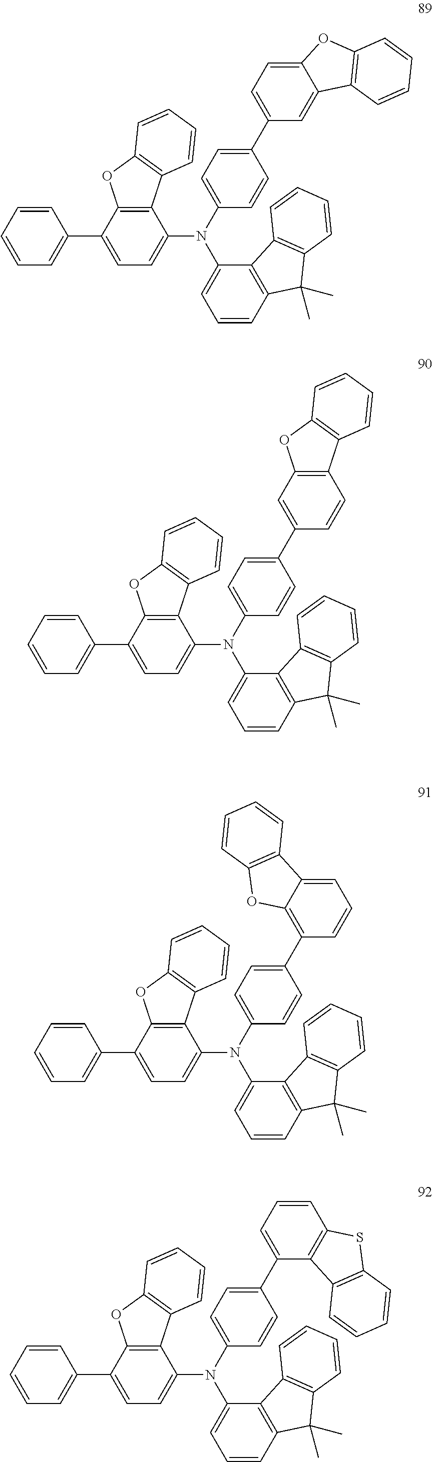 [SMILES: CC1(C)c2ccccc2-c2c(N(c3ccc(-c4ccc5oc6ccccc6c5c4)cc3)c3ccc(-c4ccccc4)c4oc5ccccc5c34)cccc21 ; CC1(C)c2ccccc2-c2c(N(c3ccc(-c4ccc5c(c4)oc4ccccc45)cc3)c3ccc(-c4ccccc4)c4oc5ccccc5c34)cccc21 ; CC1(C)c2ccccc2-c2c(N(c3ccc(-c4cccc5c4oc4ccccc45)cc3)c3ccc(-c4ccccc4)c4oc5ccccc5c34)cccc21 ; CC1(C)c2ccccc2-c2c(N(c3ccc(-c4cccc5sc6ccccc6c45)cc3)c3ccc(-c4ccccc4)c4oc5ccccc5c34)cccc21]

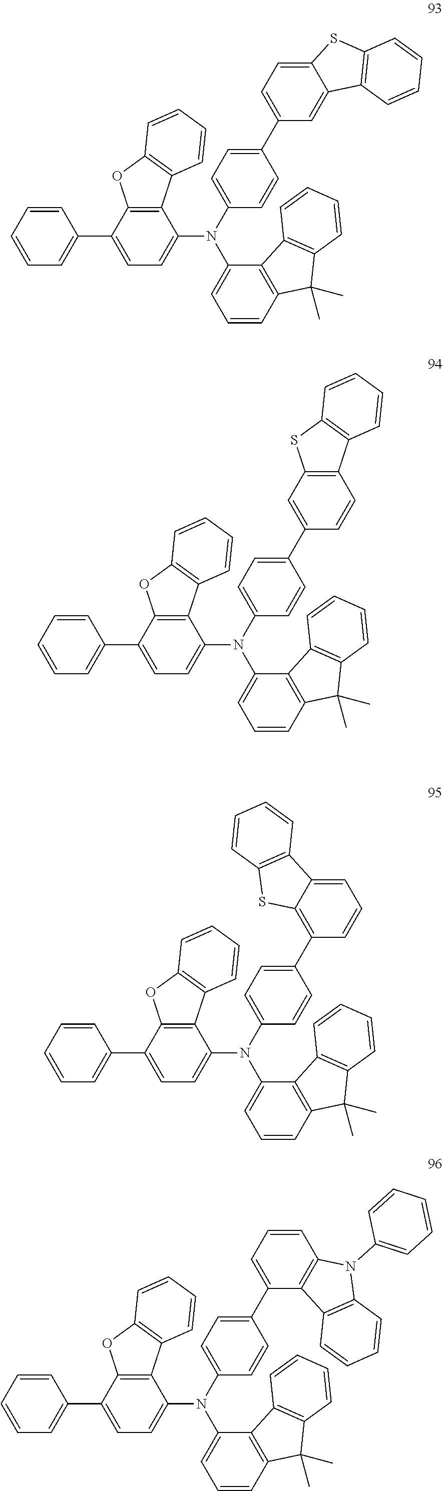 [SMILES: CC1(C)c2ccccc2-c2c(N(c3ccc(-c4ccc5sc6ccccc6c5c4)cc3)c3ccc(-c4ccccc4)c4oc5ccccc5c34)cccc21 ; CC1(C)c2ccccc2-c2c(N(c3ccc(-c4ccc5c(c4)sc4ccccc45)cc3)c3ccc(-c4ccccc4)c4oc5ccccc5c34)cccc21 ; CC1(C)c2ccccc2-c2c(N(c3ccc(-c4cccc5c4sc4ccccc45)cc3)c3ccc(-c4ccccc4)c4oc5ccccc5c34)cccc21 ; CC1(C)c2ccccc2-c2c(N(c3ccc(-c4cccc5c4c4ccccc4n5-c4ccccc4)cc3)c3ccc(-c4ccccc4)c4oc5ccccc5c34)cccc21]

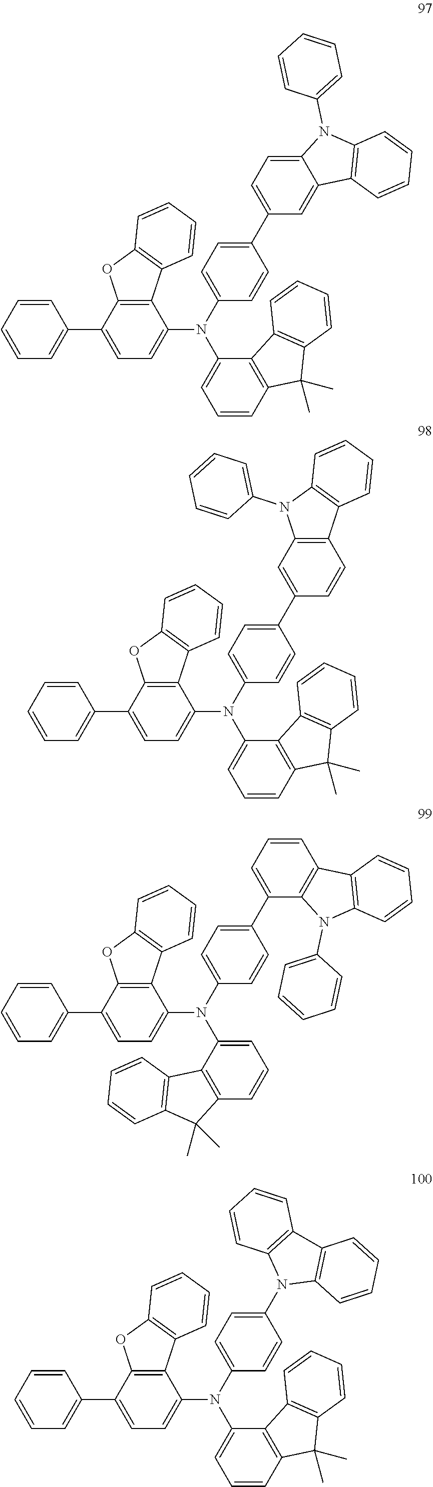

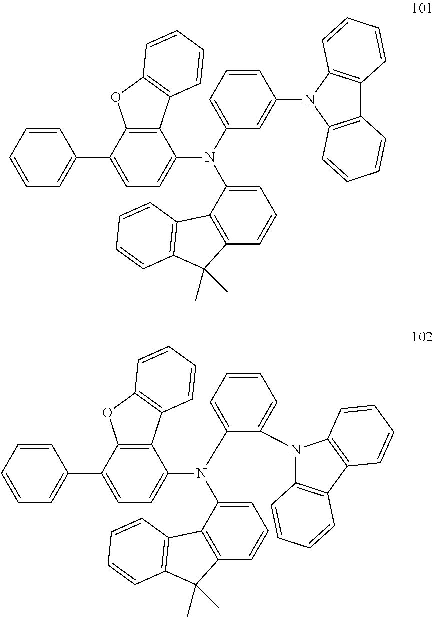 [SMILES: CC1(C)c2ccccc2-c2c(N(c3cccc(-n4c5ccccc5c5ccccc54)c3)c3ccc(-c4ccccc4)c4oc5ccccc5c34)cccc21 ; CC1(C)c2ccccc2-c2c(N(c3ccccc3-n3c4ccccc4c4ccccc43)c3ccc(-c4ccccc4)c4oc5ccccc5c34)cccc21]

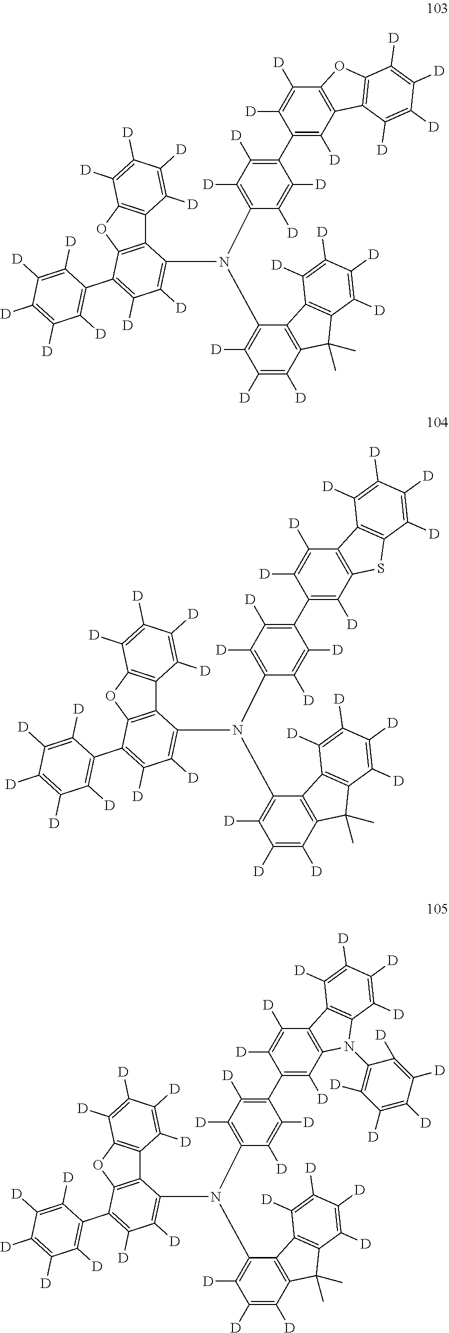

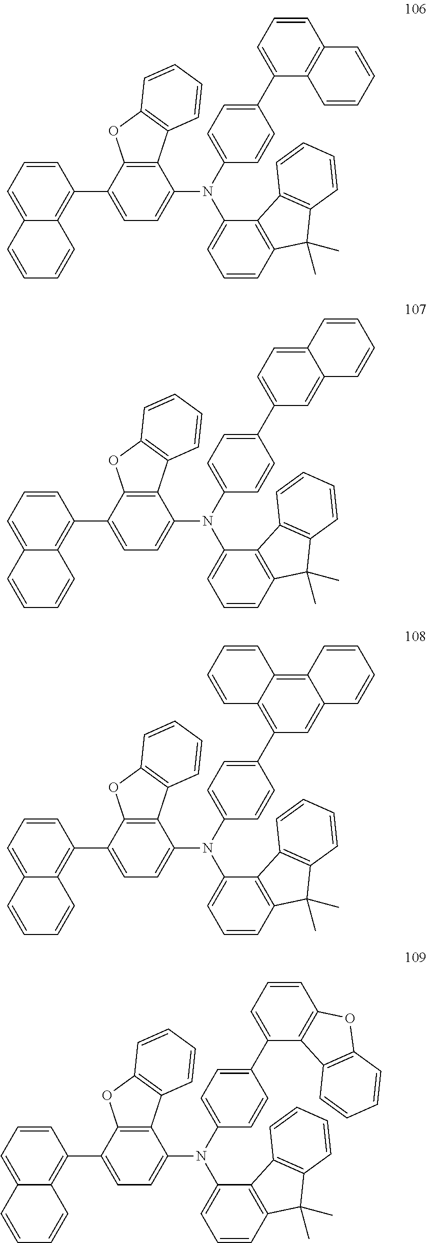

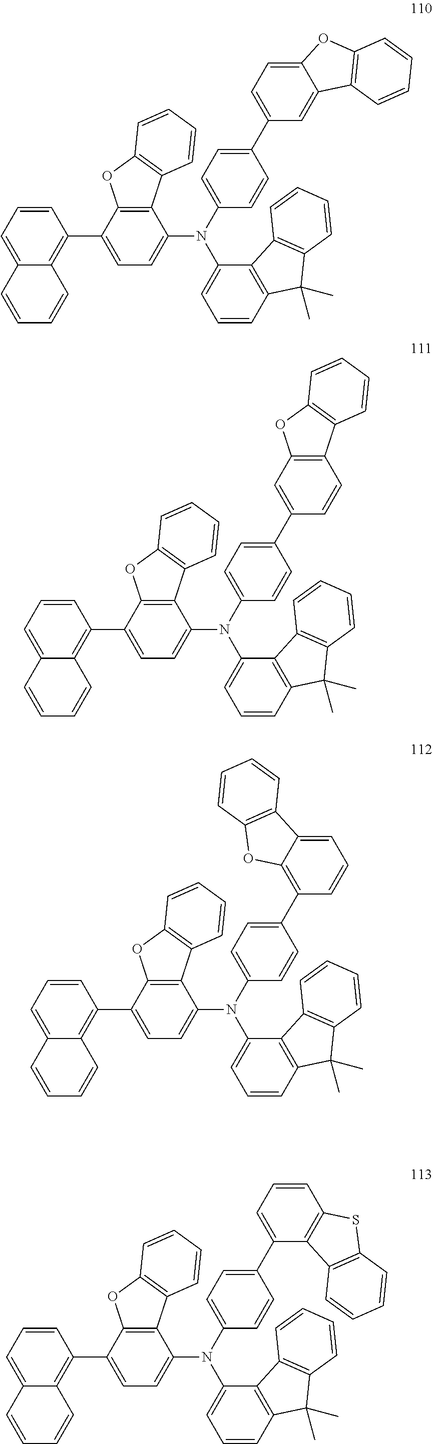

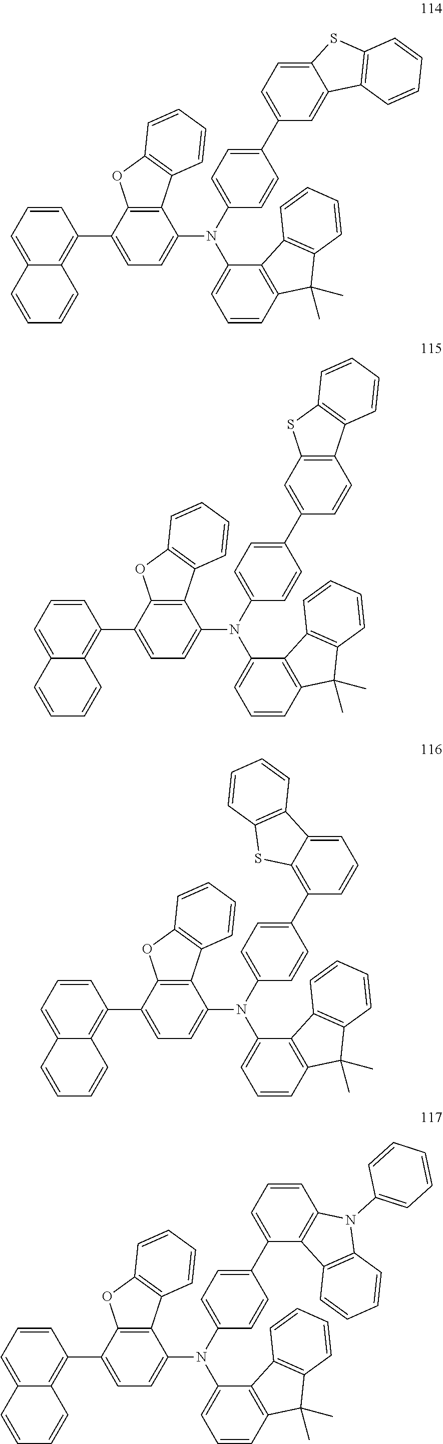

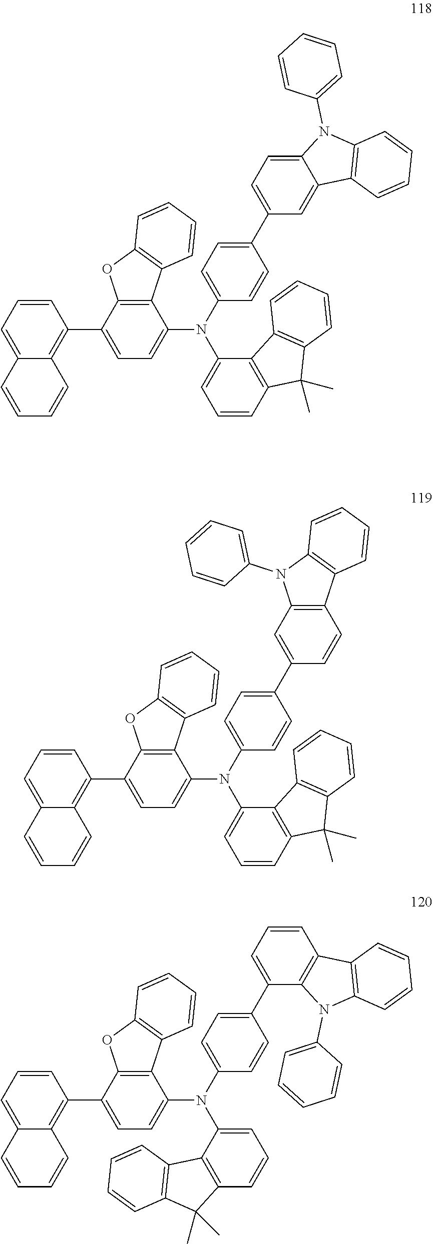 [SMILES: CC1(C)c2ccccc2-c2c(N(c3ccc(-c4ccc5c(c4)c4ccccc4n5-c4ccccc4)cc3)c3ccc(-c4cccc5ccccc45)c4oc5ccccc5c34)cccc21 ; CC1(C)c2ccccc2-c2c(N(c3ccc(-c4ccc5c6ccccc6n(-c6ccccc6)c5c4)cc3)c3ccc(-c4cccc5ccccc45)c4oc5ccccc5c34)cccc21 ; CC1(C)c2ccccc2-c2c(N(c3ccc(-c4cccc5c6ccccc6n(-c6ccccc6)c45)cc3)c3ccc(-c4cccc5ccccc45)c4oc5ccccc5c34)cccc21]

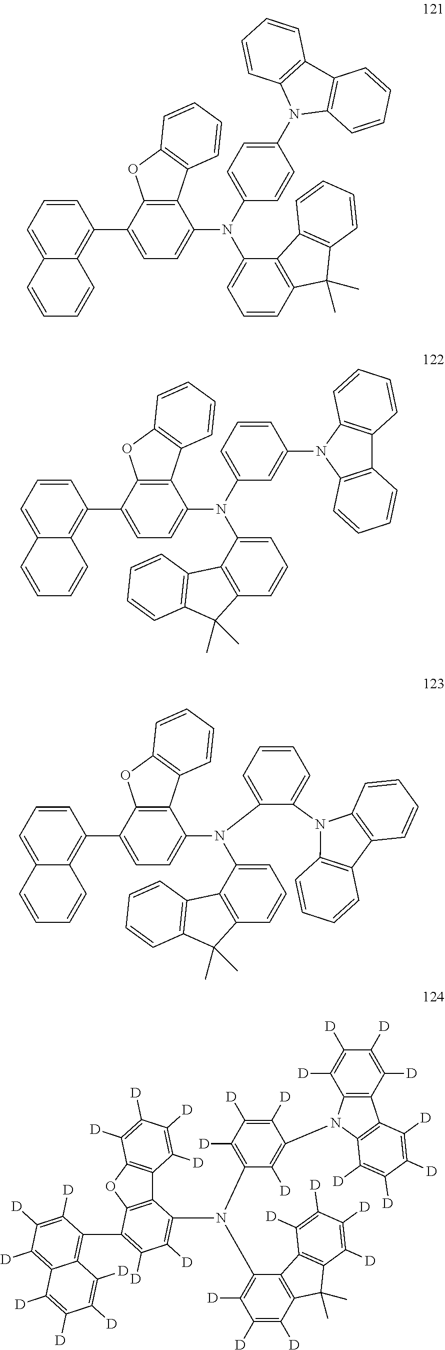

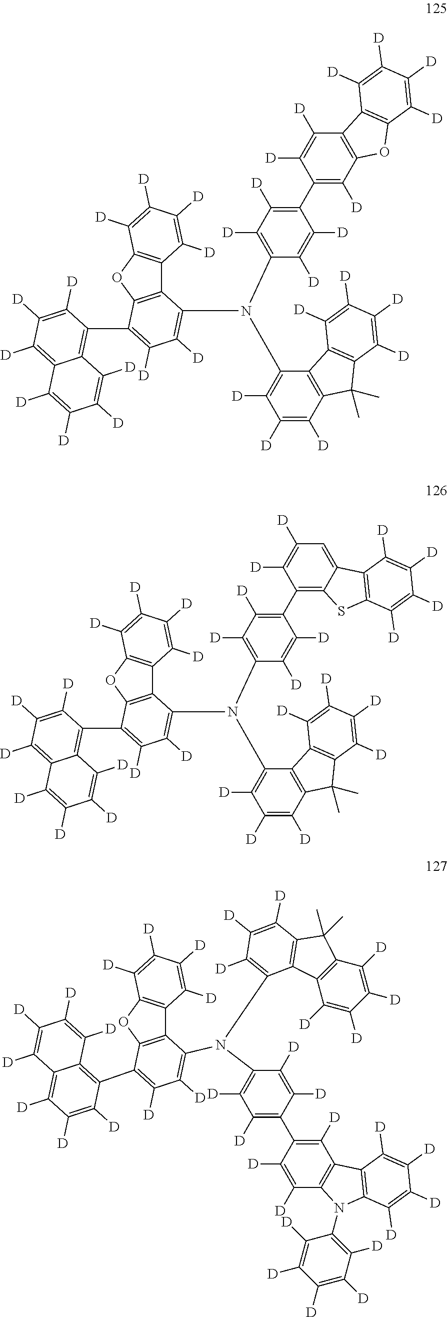

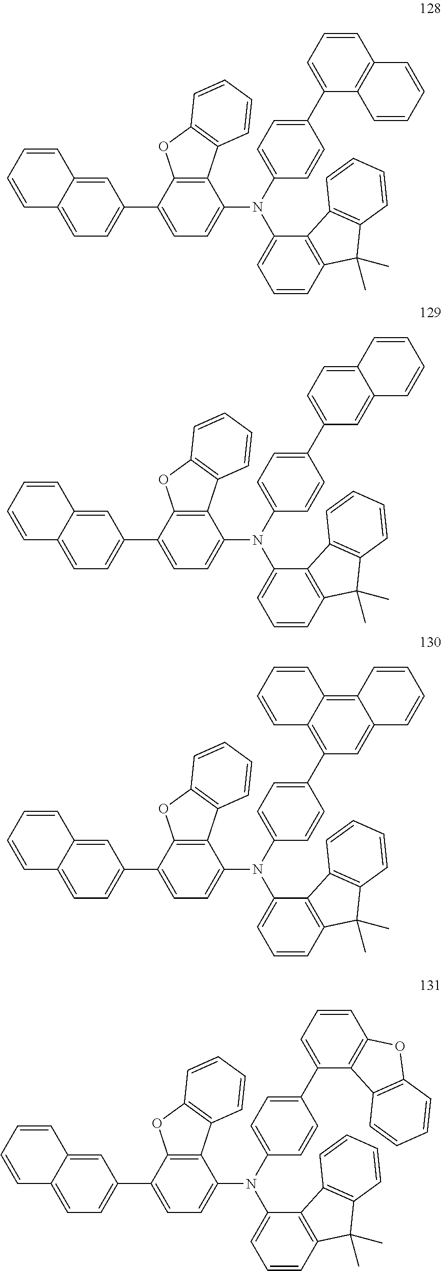

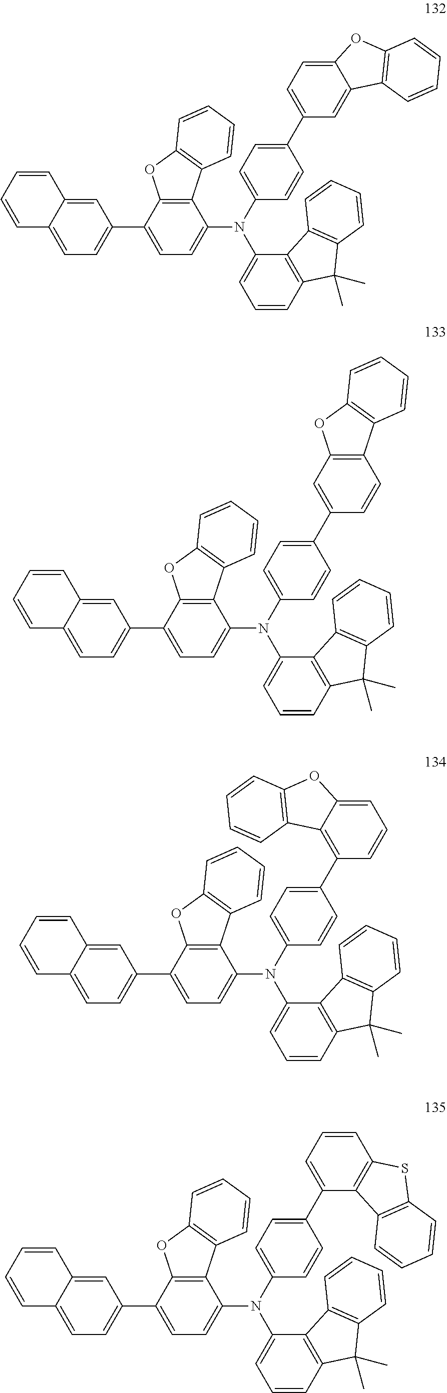 [SMILES: CC1(C)c2ccccc2-c2c(N(c3ccc(-c4ccc5oc6ccccc6c5c4)cc3)c3ccc(-c4ccc5ccccc5c4)c4oc5ccccc5c34)cccc21 ; CC1(C)c2ccccc2-c2c(N(c3ccc(-c4ccc5c(c4)oc4ccccc45)cc3)c3ccc(-c4ccc5ccccc5c4)c4oc5ccccc5c34)cccc21 ; CC1(C)c2ccccc2-c2c(N(c3ccc(-c4cccc5oc6ccccc6c45)cc3)c3ccc(-c4ccc5ccccc5c4)c4oc5ccccc5c34)cccc21 ; CC1(C)c2ccccc2-c2c(N(c3ccc(-c4cccc5sc6ccccc6c45)cc3)c3ccc(-c4ccc5ccccc5c4)c4oc5ccccc5c34)cccc21]

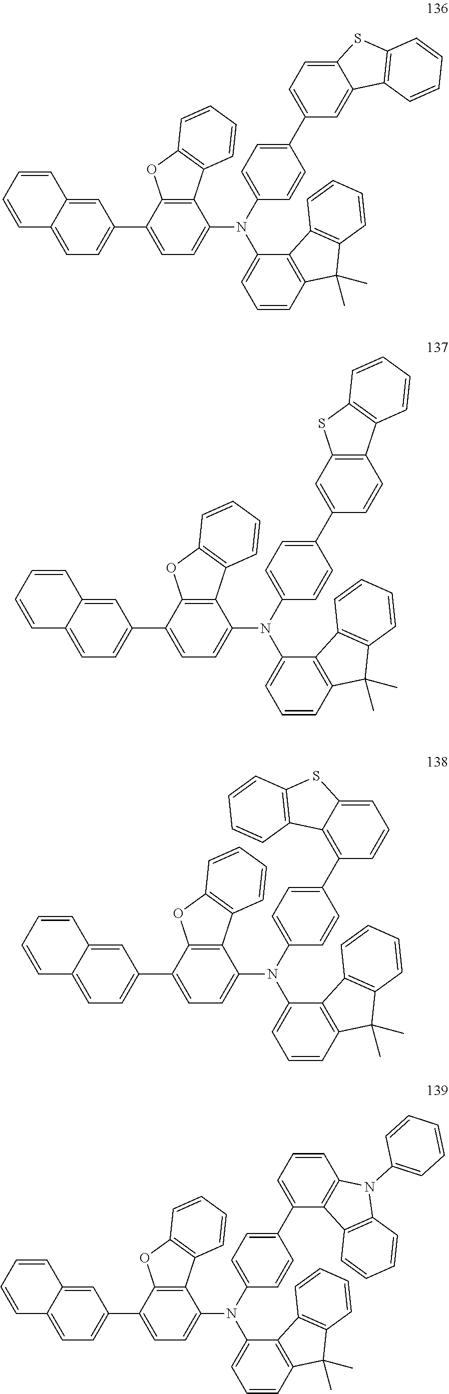 [SMILES: CC1(C)c2ccccc2-c2c(N(c3ccc(-c4ccc5sc6ccccc6c5c4)cc3)c3ccc(-c4ccc5ccccc5c4)c4oc5ccccc5c34)cccc21 ; CC1(C)c2ccccc2-c2c(N(c3ccc(-c4ccc5c(c4)sc4ccccc45)cc3)c3ccc(-c4ccc5ccccc5c4)c4oc5ccccc5c34)cccc21 ; CC1(C)c2ccccc2-c2c(N(c3ccc(-c4cccc5sc6ccccc6c45)cc3)c3ccc(-c4ccc5ccccc5c4)c4oc5ccccc5c34)cccc21 ; CC1(C)c2ccccc2-c2c(N(c3ccc(-c4cccc5c4c4ccccc4n5-c4ccccc4)cc3)c3ccc(-c4ccc5ccccc5c4)c4oc5ccccc5c34)cccc21]

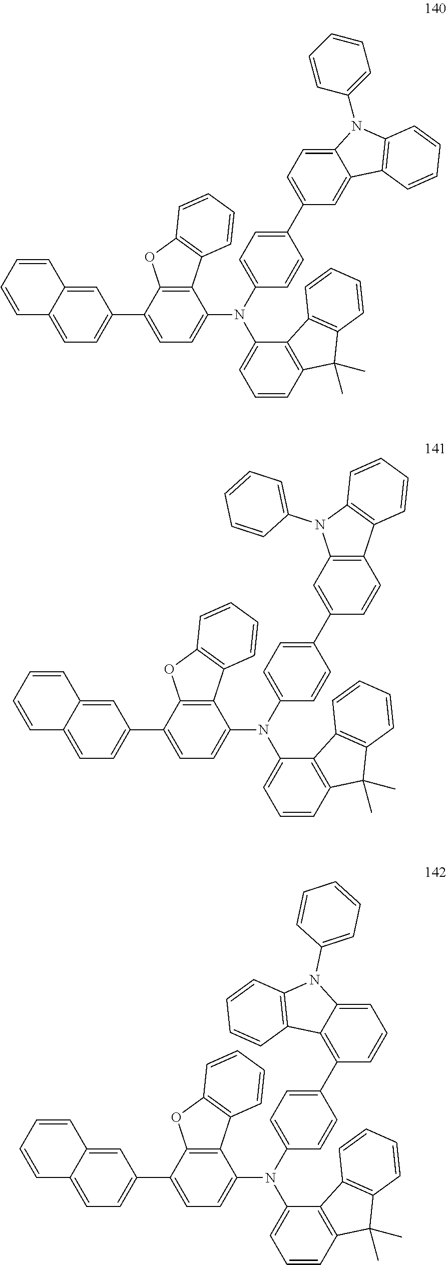 [SMILES: CC1(C)c2ccccc2-c2c(N(c3ccc(-c4ccc5c(c4)c4ccccc4n5-c4ccccc4)cc3)c3ccc(-c4ccc5ccccc5c4)c4oc5ccccc5c34)cccc21 ; CC1(C)c2ccccc2-c2c(N(c3ccc(-c4ccc5c6ccccc6n(-c6ccccc6)c5c4)cc3)c3ccc(-c4ccc5ccccc5c4)c4oc5ccccc5c34)cccc21 ; CC1(C)c2ccccc2-c2c(N(c3ccc(-c4cccc5c4c4ccccc4n5-c4ccccc4)cc3)c3ccc(-c4ccc5ccccc5c4)c4oc5ccccc5c34)cccc21]

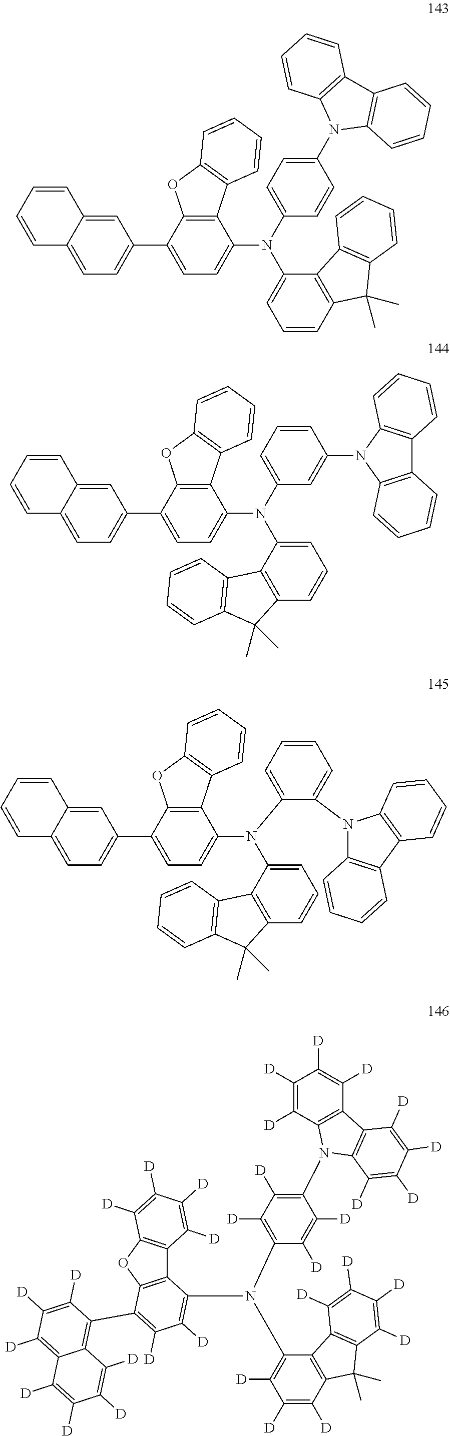

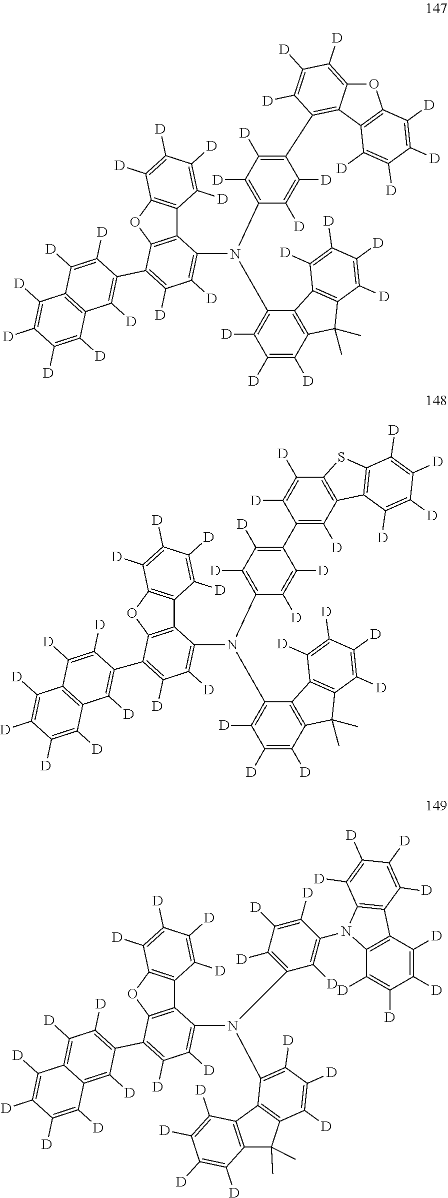

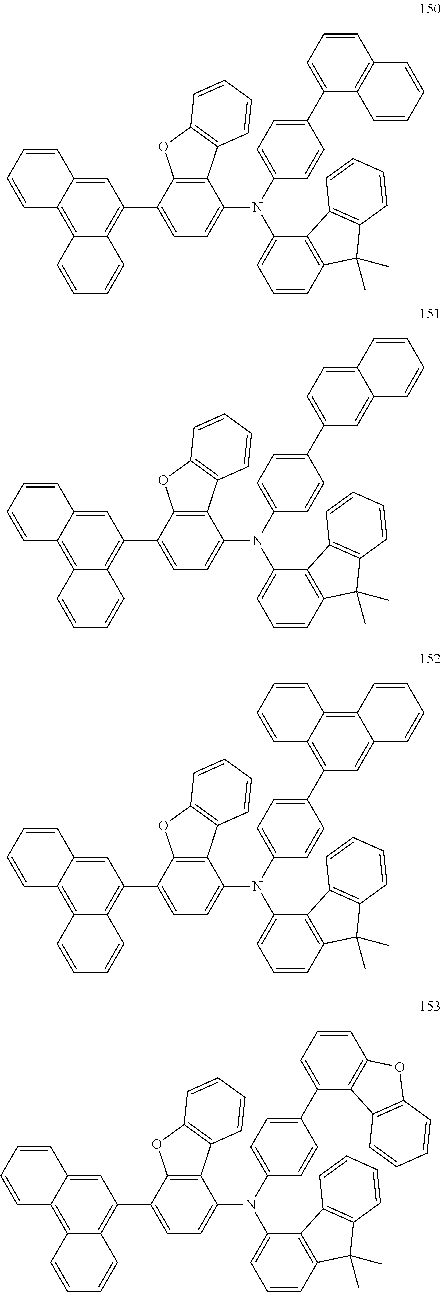 [SMILES: CC1(C)c2ccccc2-c2c(N(c3ccc(-c4cccc5ccccc45)cc3)c3ccc(-c4cc5ccccc5c5ccccc45)c4oc5ccccc5c34)cccc21 ; CC1(C)c2ccccc2-c2c(N(c3ccc(-c4ccc5ccccc5c4)cc3)c3ccc(-c4cc5ccccc5c5ccccc45)c4oc5ccccc5c34)cccc21 ; CC1(C)c2ccccc2-c2c(N(c3ccc(-c4cc5ccccc5c5ccccc45)cc3)c3ccc(-c4cc5ccccc5c5ccccc45)c4oc5ccccc5c34)cccc21 ; CC1(C)c2ccccc2-c2c(N(c3ccc(-c4cccc5oc6ccccc6c45)cc3)c3ccc(-c4cc5ccccc5c5ccccc45)c4oc5ccccc5c34)cccc21]

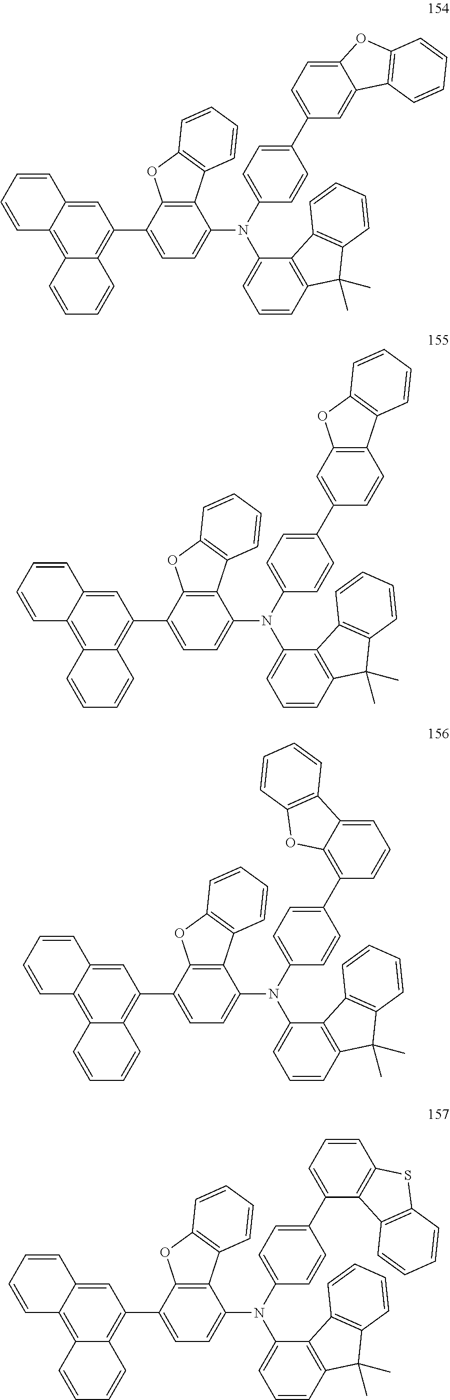

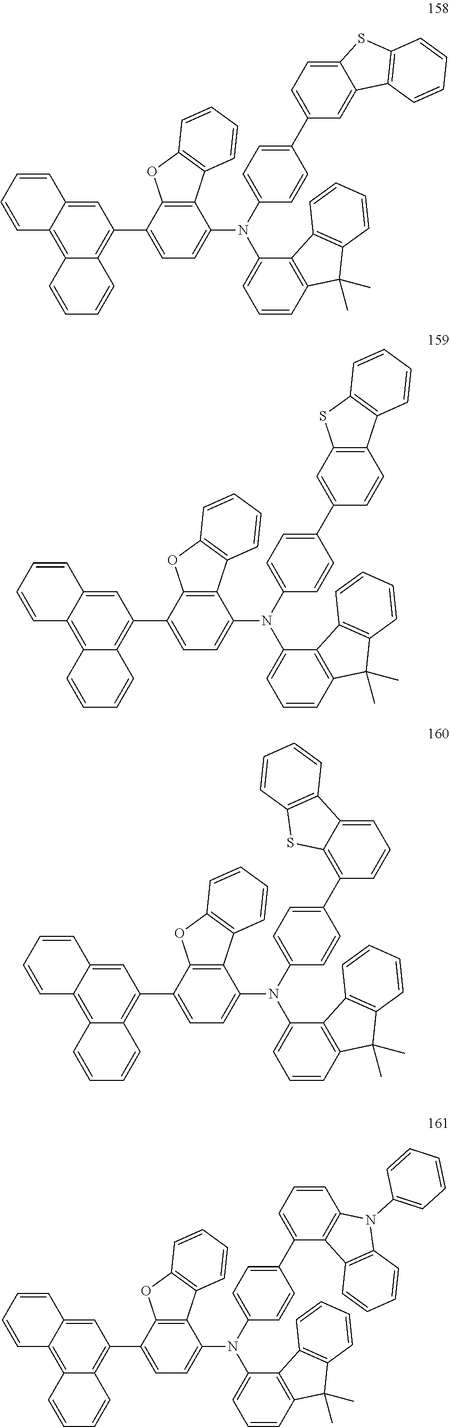

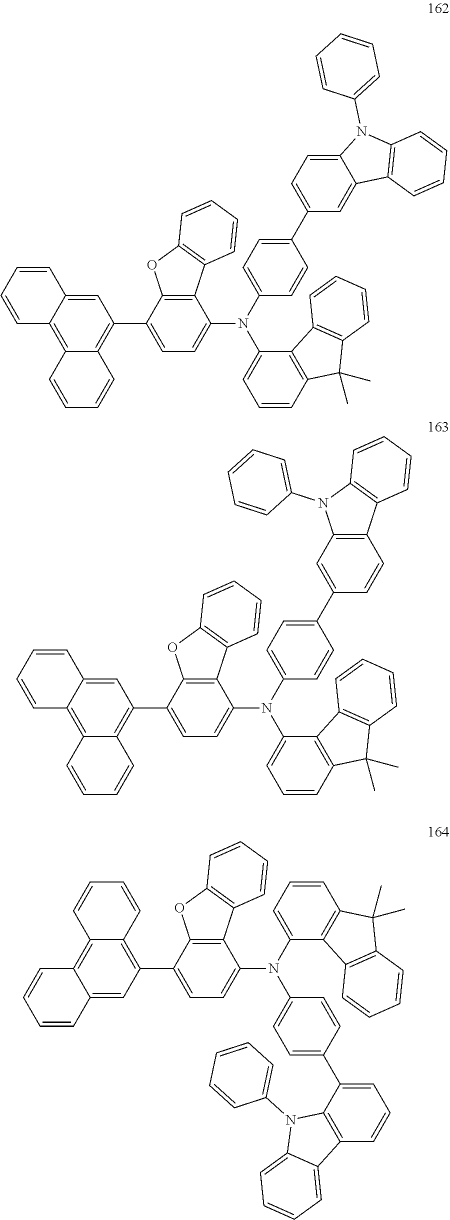

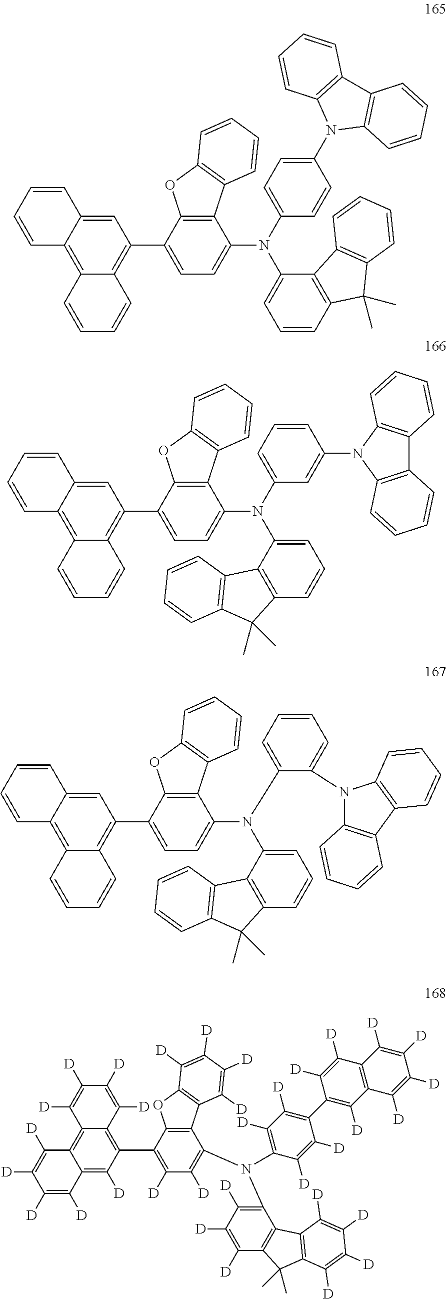

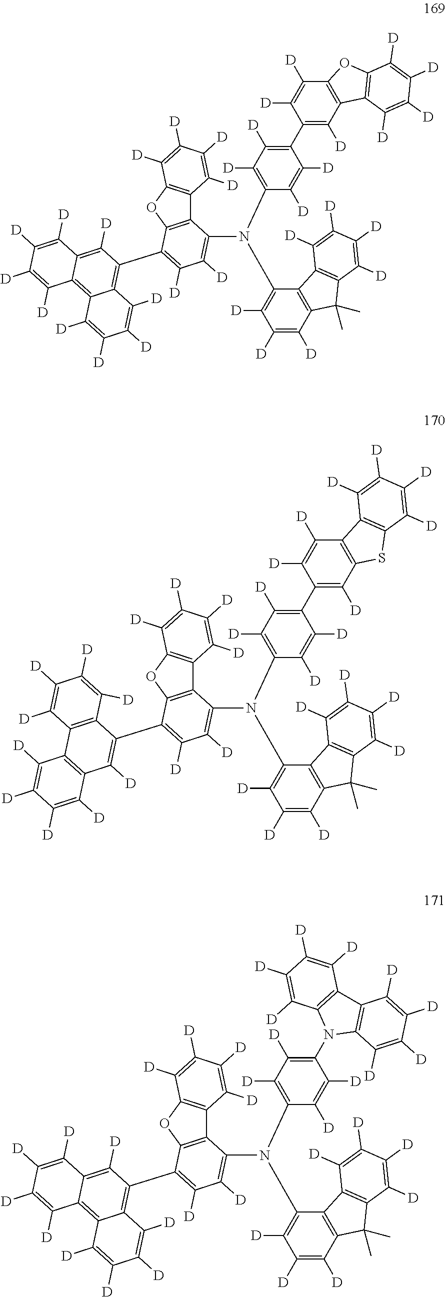

The organic light emitting diode according to an aspect of the present disclosure may include an anode and a cathode facing the anode and include an organic layer between the anode and the cathode.

According to one embodiment of the present disclosure, the organic layer includes a hole transport auxiliary layer, and the hole transport auxiliary layer may include the compound represented by Chemical Formula 1 of the present disclosure.

In addition to the hole transport auxiliary layer, the organic layer further includes one or more selected from the group consisting of a hole injection layer (HIL); a hole transport layer (HTL); an emitting layer (EML); an electron transport layer (ETL); and an electron injection layer (EIL).

For example, the organic light emitting diode may have a structure in which the anode, the hole injection layer (HIL), the hole transport layer (HTL), the hole transport auxiliary layer, the emitting layer (EML), the electron transport layer (ETL), the electron injection layer (EIL), and the cathode are stacked sequentially.

The organic layer may further include an electron transport auxiliary layer, and the like.

The anode may include a material, such as indium tin oxide (ITO), indium zinc oxide (IZO), tin oxide (SnO₂), or zinc oxide (ZnO), that is transparent and has excellent conductivity.

The cathode may include a material, such as lithium (Li), aluminum (Al), aluminum-lithium (Al—Li), calcium (Ca), magnesium (Mg), magnesium-indium (Mg—In), or magnesium-silver (Mg—Ag). In addition, in the case of a top emission organic light emitting diode, a transparent cathode through which light may transmit may be formed by using ITO or IZO.

A capping layer CPL may be formed on a surface of the cathode using a composition for the formation of the capping layer.

The compound for the hole injection layer or the hole transport layer is not specifically limited, and any compound may be used as long as it is generally used as the compound for the hole injection layer or the hole transport layer. Non-limiting examples of the compound for the hole injection layer or the hole transport layer include phthalocyanine derivatives, porphyrin derivatives, triarylamine derivatives, and indolocarbazole derivatives. For example, the compound for the hole injection layer or the hole transport layer includes 1,4,5,8,9,11-hexaazatriphenylene-hexacarbonitrile (HAT-CN), copper phthalocyanine (CuPc), 4,4′,4″-tris(3-methylphenylamino)triphenylamine (m-MTDATA), 4,4′,4″-tris(3-methylphenylamino) phenoxybenzene (m-MTDAPB), 4,4′,4″-tri (N-carbazolyl)triphenylamine (TCTA), 4,4′,4″-tris(N-(2-naphthyl)-N-phenylamino)-triphenylamine (2-TNATA),N4,N4,N4′,N4′-tetra([1,1′-biphenyl]-4-yl)-[1,1′-biphenyl]-4,4′-diamine, bis(N-(1-naphthyl-n-phenyl))benzidine(α-NPD), N,N′-di(naphthalen-1-yl)-N,N′-biphenyl-benzidine(NPB) or N,N′-biphenyl-N,N′-bis(3-methylphenyl)-1,1′-biphenyl-4,4′-diamine (TPD), and the like.

The compound included in the emitting layer is not specifically limited, and any compound may be used as long as it is generally used as a compound for the emitting layer. A single light-emitting compound or a light-emitting host compound may be used.

Although examples of the light-emitting compound in the emitting layer include compounds capable of emitting light through phosphorescence, fluorescence, thermally activated delayed fluorescence (TADF) (also referred to as E-type delayed fluorescence), triplet-triplet quenching, or combinations of these processes, the present disclosure is not limited thereto. Emitting compounds may be selected from various materials depending on the desired emitting color. Non-limiting examples of the light-emitting compounds may include condensed ring derivatives, such as phenanthrene, anthracene, pyrene, tetracene, pentacene, perylene, naphthopyrene, dibenzopyrene, rubrene, and chrysene, benzoxazole derivatives, benzothiazole derivatives, benzoimidazole derivatives, benzotriazole derivatives, oxazole derivatives, oxadiazole derivatives, thiazole derivatives, imidazole derivatives, thiadiazole derivatives, triazole derivatives, pyrazoline derivatives, stilbene derivatives, thiophene derivatives, tetraphenylbutadiene derivatives, cyclopentadiene derivatives, bistyryl derivatives, bistyrylarylene derivatives, diazynedacene derivatives, furan derivatives, benzofuran derivatives, isobenzofuran derivatives, dibenzofuran derivatives, coumarin derivatives, dicyanomethylenepyran derivatives, dicyanomethylenethiopyran derivatives, polymethine derivatives, cyanine derivatives, oxobenzoanthracene derivatives, xanthene derivatives, rhodamine derivatives, fluorescein derivatives, pyrylium derivatives, carbostyryl derivatives, acridine derivatives, oxazine derivatives, phenylene oxide derivatives, quinacridone derivatives, quinazoline derivatives, pyrrolopyridine derivatives, furopyridine derivatives, 1,2,5-thiadiazolopyrene derivatives, pyromethene derivatives, perrinone derivatives, pyrrolopyrrole derivatives, scoo arylium derivatives, biolanthrone derivatives, phenazine derivatives, acridone derivatives, deazaflavin derivatives, fluorene derivatives, benzofluorene derivatives, aromatic boron derivatives, aromatic nitrogen boron derivatives, metal complexes (complexes in which metals, such as Ir, Pt, Au, Eu, Ru, Re, Ag, and Cu, are bonded to heteroaromatic ring ligands). For example, the light-emitting compound includes N1,N1,N6,N6-tetrakis(4-(1-silyl)phenyl) pyrene-1,6-diamine, 2,12-di-tert-butyl-5,9-bis(4-(tert-butyl)phenyl)-7-(3,5-di-tert-butylphenyl)-5,9-dihydro-5,9-diaza-13b-boranaphtho[3,2,1-de]anthracene(t-DABNA-dtB), Platinum octaethylporphyrin (PtOEP), Ir(ppy)₃, Ir(ppy)₂(acac), Ir(mppy)₃, Ir(PPy)₂ (m-bppy), BtpIr(acac), Ir(btp)₂(acac), Ir(2-phq)₃, Hex-Ir(phq)₃, Ir(fbi)₂(acac), fac-Tris(2-(3-p-xylyl)phenyl)pyridine iridium(III), Eu(dbm)₃(Phen), Ir(piq)₃, Ir(piq)₂(acac), Ir(Fliq)₂(acac), Ir(Flq)₂(acac), Ru(dtb-bpy)3·2(PF6), Ir(BT)₂(acac), Ir(DMP)₃, Ir(Mphq)₃IR (phq)₂tpy, fac-Ir(ppy)₂Pc, Ir(dp)PQ₂, Ir(Dpm)(Piq)₂, Hex-Ir(piq)₂ (acac), Hex-Ir(piq)₃, Ir(dmpq)₃, Ir(dmpq)₂(acac), FPQIrpic, FIrpic, and the like.

As the host compound of the emitting layer, a light-emitting host, a hole transporting host, an electron transporting host, or combinations thereof may be used. Non-limiting examples of compounds for the light-emitting host may include condensed ring derivatives, such as anthracene and pyrene, bistyryl derivatives, such as bistyryl anthracene derivatives and distyrylbenzene derivatives, tetraphenylbutadiene derivatives, cyclopentadiene derivatives, fluorene derivatives, benzofluorene derivatives, N-phenylcarbazole derivatives, and carbazonitrile derivatives. Non-limiting examples of the material for the hole transporting host may include carbazole derivatives, dibenzofuran derivatives, dibenzothiophene derivatives, triarylamine derivatives, indolocarbazole derivatives, and benzoxazinophenoxazine derivatives. Non-limiting examples of the material for the electron transporting host may include pyridine derivatives, triazine derivatives, phosphine oxide derivatives, benzofuropyridine derivatives, and dibenzoxacillin derivatives. The material for the electron transporting host includes, for example, 9,10-bis(2-naphthyl) anthracene (ADN), tris(8-hydroxyquinolinolato)aluminum(Alq₃), Balq(8-hydroxyquinoline beryllium salt), DPVBi (4,4′-bis(2,2-biphenylethenyl)-1,1′biphenyl) series, spiro-DPVBi(spiro-4,4′bis(2,2-biphenylethenyl)-1,1′biphenyl), LiPBO(2-(2-benzooxazolyl)-phenol lithium salt), bis(biphenylvinyl)benzene, aluminum-quinoline metal complex, imidazole, thiazole and oxazole metal complexes, and the like.

The compound for the electron injection layer or the electron transport layer is not specifically limited, and any compound may be used as long as it is generally used as the compound for the electron injection layer or the electron transport layer. Non-limiting examples of the compound for the electron injection layer or the electron transport layer may include pyridine derivatives, naphthalene derivatives, anthracene derivatives, phenanthroline derivatives, perinone derivatives, coumarin derivatives, naphthalimide derivatives, anthraquinone derivatives, diphenoquinone derivatives, phenylquinone derivatives, perylene derivatives, oxadiazole derivatives, thiophene derivatives, triazole derivatives, thiadiazole derivatives, metal complexes of oxin derivatives, quinolinol-based metal complexes, quinoxaline derivatives, polymers of quinoxaline derivatives, benzazoles compounds, gallium complexes, pyrazole derivatives, perfluorinated phenylene derivatives, triazine derivatives, pyrazine derivatives, benzoquinoline derivatives, imidazopyridine derivatives, borane derivatives, benzoimidazole derivatives, benzoxazole derivatives, benzothiazole derivatives, quinoline derivatives, oligopyridine derivatives such as terpyridine, bipyridine derivatives, terpyridine derivatives, naphthyridine derivatives, aldazine derivatives, carbazole derivatives, indole derivatives, phosphine oxide derivatives, bistyryl derivatives, quinolinol-based metal complexes, hydroxyazole-based metal complexes, azomethine-based metal complexes, tropolone-based metal complexes, flavonol-based metal complexes, benzoquinoline-based metal complexes, metal salts, and the like. The materials may be used alone, but may also be used by being mixed with other materials. The compound for the electron injection layer or the electron transport layer may include, for example, materials, such as 2-(4-9,10-di(naphthalen-2-yl)anthracen-2-yl)phenyl)-1-phenyl-1H-benzo[d]imidazole, tris(8-hydroxyquinolinolato)aluminum (Alq₃), LiF, Liq, Li₂O, BaO, NaCl, and CsF.

An electron transport auxiliary layer may be formed between the electron transport layer and the emitting layer. The compound for the hole blocking layer is not specifically limited, and any compound may be used as long as it is generally used as the compound for the electron transport auxiliary layer. For example, the electron transport auxiliary layer may include pyrimidine derivatives and the like.

The organic light emitting diode according to one embodiment of the present disclosure may be a top emission device or a bottom emission device.

The organic light emitting diode according to one embodiment of the present disclosure may be used in display devices.

The organic light emitting diode according to the embodiment of the present disclosure may be applied to transparent display devices, mobile display devices, flexible display devices, and the like, but is not limited thereto.

Hereinafter, a synthesis method and examples of the compounds will be described with representative examples. However, the method of synthesizing the compounds according to the present disclosure is not limited to an exemplary method below, or the performance of the present disclosure is not limited to examples below.

### Synthesis Example

### 1. Synthesis of Product 1

Product 1 may be synthesized as follows, but is not limited thereto.

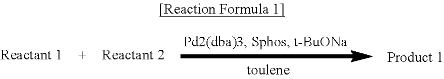

Reactant 1 (35.88 mmol) of P1, Reactant 2 (35.17 mmol) of P1, t-BuONa (70.3 mmol), Pd₂(dba)₃ (0.7 mmol), Sphos (1.41 mmol), and toluene were added to a reaction flask, stirred, and refluxed under nitrogen atmosphere. After the reaction was finished, an organic layer was extracted by using toluene and water. The extracted solution was treated with MgSO₄ to remove remaining moisture, concentrated under reduced pressure, purified by using a column chromatography method, and then recrystallized to obtain Product 1 of P1. The synthesized result of Product 1 of P1 is as shown in Table 1 below.

In addition, except that Reactants 1 and Reactants 2 of P2 to P19 in Table 1 below were used instead of Reactant 1 and Reactant 2 of P1 in the method of synthesizing the product, products of P2 to P19 were synthesized in the same method, and the synthesized result is as shown in Table 1 below.

TABLE 1

| | | | | Ac- | |
|---|---|---|---|---|---|
| | | | | quired | |
| | | | | amount | |
| | | | | (Ac- | |
| | | | Product | quired | [M + |
| Items | Reactant 1 | Reactant 2 | 1 | rate) | H]⁺ |
| P1 | 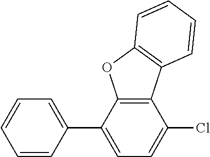 [SMILES: Clc1ccc(-c2ccccc2)c2oc3ccccc3c12] | 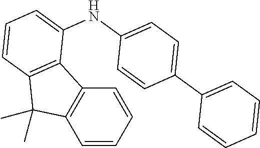 [SMILES: CC1(C)c2ccccc2-c2c(Nc3ccc(-c4ccccc4)cc3)cccc21] | Com- pound 4 | 18.9 g (89%) | 603.77 |
| P2 | 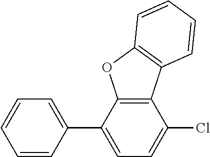 [SMILES: Clc1ccc(-c2ccccc2)c2oc3ccccc3c12] | 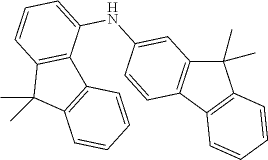 [SMILES: CC1(C)c2ccccc2-c2ccc(Nc3cccc4c3-c3ccccc3C4(C)C)cc21] | Com- pound 6 | 19.5 g (86%) | 643.83 |
| P3 | 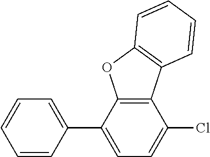 [SMILES: Clc1ccc(-c2ccccc2)c2oc3ccccc3c12] | 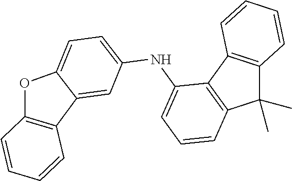 [SMILES: CC1(C)c2ccccc2-c2c(Nc3ccc4oc5ccccc5c4c3)cccc21] | Com- pound 11 | 18.9 g (87%) | 617.24 |
| P4 | 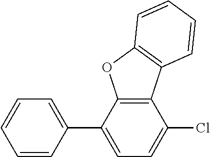 [SMILES: Clc1ccc(-c2ccccc2)c2oc3ccccc3c12] | 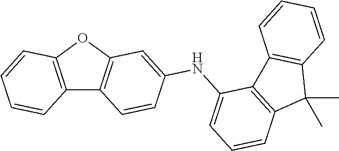 [SMILES: CC1(C)c2ccccc2-c2c(Nc3ccc4c(c3)oc3ccccc34)cccc21] | Com- pound 10 | 18.3 g (84%) | 617.24 |
| P5 | 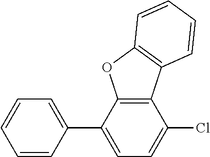 [SMILES: Clc1ccc(-c2ccccc2)c2oc3ccccc3c12] | 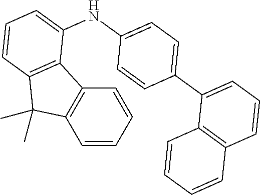 [SMILES: CC1(C)c2ccccc2-c2c(Nc3ccc(-c4cccc5ccccc45)cc3)cccc21] | Com- pound 85 | 18.4 g (80%) | 653.27 |
| P6 | 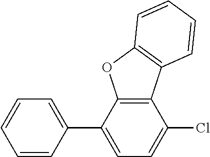 [SMILES: Clc1ccc(-c2ccccc2)c2oc3ccccc3c12] | 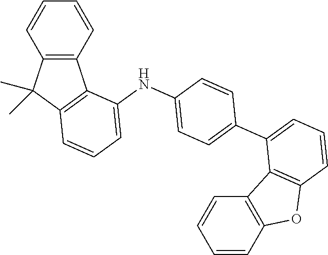 [SMILES: CC1(C)c2ccccc2-c2c(Nc3ccc(-c4cccc5oc6ccccc6c45)cc3)cccc21] | Com- pound 89 | 20 g (82%) | 693.27 |
| P7 | 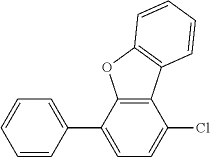 [SMILES: Clc1ccc(-c2ccccc2)c2oc3ccccc3c12] | 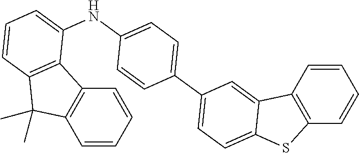 [SMILES: CC1(C)c2ccccc2-c2c(Nc3ccc(-c4ccc5sc6ccccc6c5c4)cc3)cccc21] | Com- pound 93 | 21.5 g (86%) | 709.24 |
| P8 | 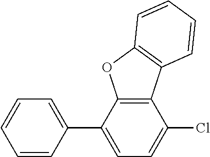 [SMILES: Clc1ccc(-c2ccccc2)c2oc3ccccc3c12] | 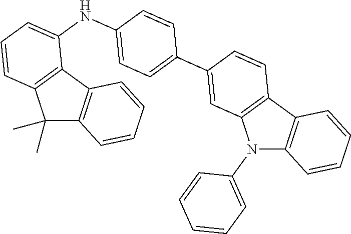 [SMILES: CC1(C)c2ccccc2-c2c(Nc3ccc(-c4ccc5c6ccccc6n(-c6ccccc6)c5c4)cc3)cccc21] | Com- pound 98 | 21.6 g (80%) | 768.31 |
| P9 | 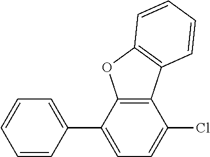 [SMILES: Clc1ccc(-c2ccccc2)c2oc3ccccc3c12] | 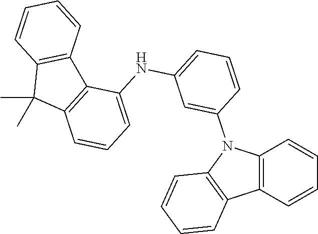 [SMILES: CC1(C)c2ccccc2-c2c(Nc3cccc(-n4c5ccccc5c5ccccc54)c3)cccc21] | Com- pound 101 | 19.7 g (81%) | 692.28 |
| P10 | 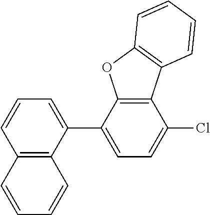 [SMILES: Clc1ccc(-c2cccc3ccccc23)c2oc3ccccc3c12] | 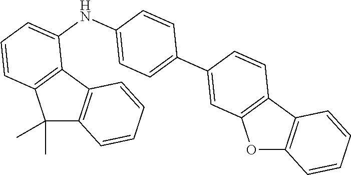 [SMILES: CC1(C)c2ccccc2-c2c(Nc3ccc(-c4ccc5c(c4)oc4ccccc45)cc3)cccc21] | Com- pound 111 | 22.5 g (86%) | 743.28 |
| P11 | 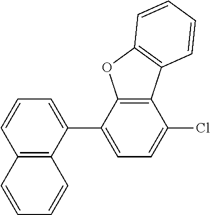 [SMILES: Clc1ccc(-c2cccc3ccccc23)c2oc3ccccc3c12] | 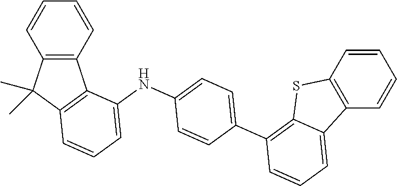 [SMILES: CC1(C)c2ccccc2-c2c(Nc3ccc(-c4cccc5c4sc4ccccc45)cc3)cccc21] | Com- pound 116 | 23.3 g (87%) | 759.26 |
| P12 | 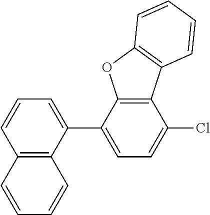 [SMILES: Clc1ccc(-c2cccc3ccccc23)c2oc3ccccc3c12] | 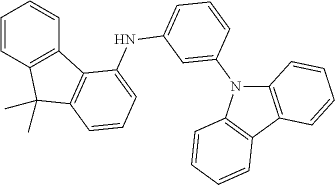 [SMILES: CC1(C)c2ccccc2-c2c(Nc3cccc(-n4c5ccccc5c5ccccc54)c3)cccc21] | Com- pound 122 | 21.2 g (81%) | 742.30 |
| P13 | 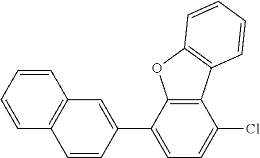 [SMILES: Clc1ccc(-c2ccc3ccccc3c2)c2oc3ccccc3c12] | 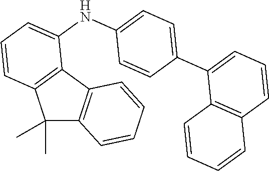 [SMILES: CC1(C)c2ccccc2-c2c(Nc3ccc(-c4cccc5ccccc45)cc3)cccc21] | Com- pound 128 | 20.3 g (82%) | 703.29 |
| P14 | 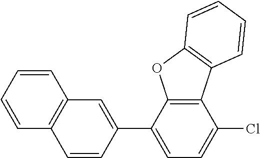 [SMILES: Clc1ccc(-c2ccc3ccccc3c2)c2oc3ccccc3c12] | 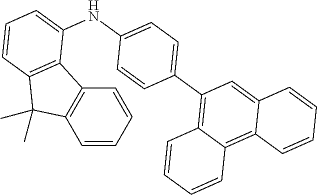 [SMILES: CC1(C)c2ccccc2-c2c(Nc3ccc(-c4cc5ccccc5c5ccccc45)cc3)cccc21] | Com- pound 130 | 20.7 g (78%) | 753.30 |
| P15 | 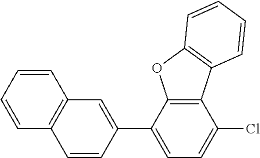 [SMILES: Clc1ccc(-c2ccc3ccccc3c2)c2oc3ccccc3c12] | 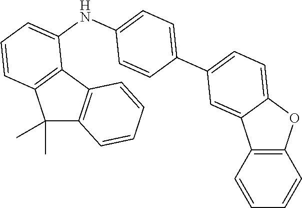 [SMILES: CC1(C)c2ccccc2-c2c(Nc3ccc(-c4ccc5oc6ccccc6c5c4)cc3)cccc21] | Com- pound 132 | 20.9 g (80%) | 743.28 |
| P16 | 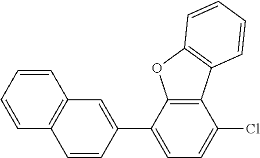 [SMILES: Clc1ccc(-c2ccc3ccccc3c2)c2oc3ccccc3c12] | 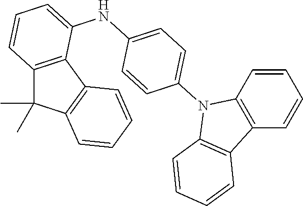 [SMILES: CC1(C)c2ccccc2-c2c(Nc3ccc(-n4c5ccccc5c5ccccc54)cc3)cccc21] | Com- pound 143 | 21.7 g (83%) | 742.30 |
| P17 | 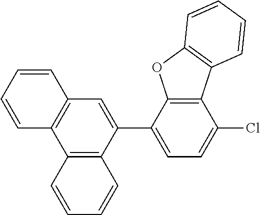 [SMILES: Clc1ccc(-c2cc3ccccc3c3ccccc23)c2oc3ccccc3c12] | 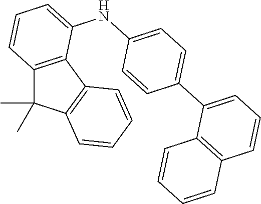 [SMILES: CC1(C)c2ccccc2-c2c(Nc3ccc(-c4cccc5ccccc45)cc3)cccc21] | Com- pound 150 | 21.7 g (82%) | 753.30 |
| P18 | 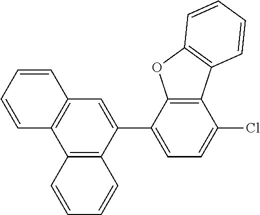 [SMILES: Clc1ccc(-c2cc3ccccc3c3ccccc23)c2oc3ccccc3c12] | 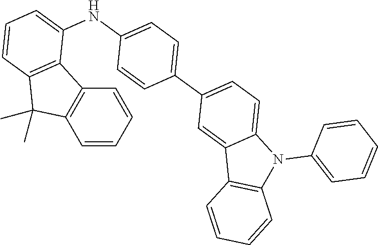 [SMILES: CC1(C)c2ccccc2-c2c(Nc3ccc(-c4ccc5c(c4)c4ccccc4n5-c4ccccc4)cc3)cccc21] | Com- pound 162 | 22.5 g (82%) | 868.35 |
| P19 | 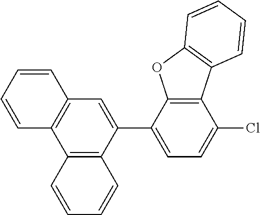 [SMILES: Clc1ccc(-c2cc3ccccc3c3ccccc23)c2oc3ccccc3c12] | 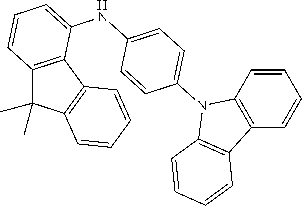 [SMILES: CC1(C)c2ccccc2-c2c(Nc3ccc(-n4c5ccccc5c5ccccc54)cc3)cccc21] | Com- pound 165 | 22.6 g (74%) | 792.31 |

### 2. Deuterium Substitution and Synthesis of Final Product

A method of substituting deuterium of Product 2 according to the present disclosure may be performed as in Reaction Formula 2 below, but is not limited thereto.

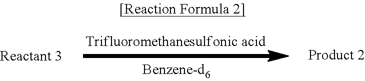

Reactant 3 (10 mmol) of P20 and trifluoromethanesulfonic acid (0.05 mmol) were dissolved in Benzene-d₆, stirred at 70° C. for 12 hours, and then cooled to room temperature. Then, after neutralization with an aqueous K₃PO₄ solution, the organic layer was extracted using dichloromethane and water. The extracted solution was treated with MgSO₄ to remove remaining moisture, concentrated under reduced pressure, purified by using the column chromatography method, and then recrystallized to obtain Product 2 of P20. The synthesized result of Product 2 of P20 is as shown in Table 2 below. In addition, except that Reactants 3 of P21 to P31 in Table 2 below were used instead of Reactant 3 of P20 in the method of substituting deuterium of product 2, Products 2 of P21 to P31 were synthesized by substituting deuterium in the same method, and the synthesized result is as shown in Table 2 below.

TABLE 2

| | | | Acquired | | |
|---|---|---|---|---|---|
| | | | amount | Deuterium | |
| | | | (Acquired | substitution | |
| Items | Reactant 3 | Product 2 | rate | rate | [M + H]⁺ |
| P20 | 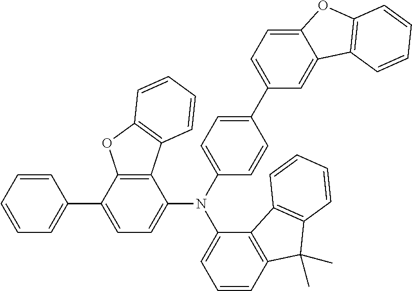 [SMILES: CC1(C)c2ccccc2-c2c(N(c3ccc(-c4ccc5oc6ccccc6c5c4)cc3)c3ccc(-c4ccccc4)c4oc5ccccc5c34)cccc21] | Compound 103 | 5.93 g (82%) | 91% | 722.45 |
| P21 | 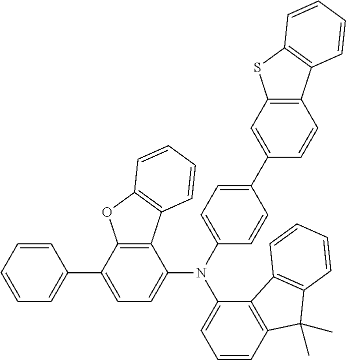 [SMILES: CC1(C)c2ccccc2-c2c(N(c3ccc(-c4ccc5c(c4)sc4ccccc45)cc3)c3ccc(-c4ccccc4)c4oc5ccccc5c34)cccc21] | Compound 104 | 5.99 g (81%) | 90% | 738.43 |
| P22 | 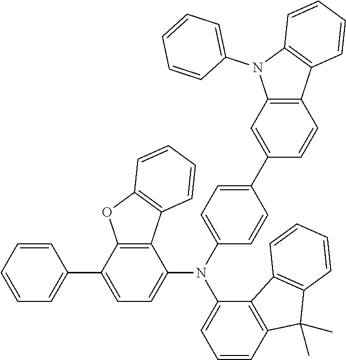 [SMILES: CC1(C)c2ccccc2-c2c(N(c3ccc(-c4ccc5c6ccccc6n(-c6ccccc6)c5c4)cc3)c3ccc(-c4ccccc4)c4oc5ccccc5c34)cccc21] | Compound 105 | 6.26 g (78%) | 84% | 802.53 |
| P23 | 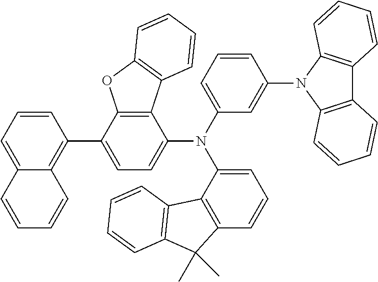 [SMILES: CC1(C)c2ccccc2-c2c(N(c3cccc(-n4c5ccccc5c5ccccc54)c3)c3ccc(-c4cccc5ccccc45)c4oc5ccccc5c34)cccc21] | Compound 124 | 6.12 g (79%) | 83% | 774.50 |
| P24 | 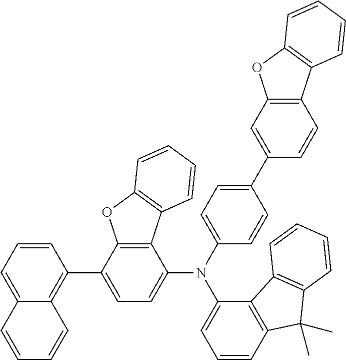 [SMILES: CC1(C)c2ccccc2-c2c(N(c3ccc(-c4ccc5c(c4)oc4ccccc45)cc3)c3ccc(-c4cccc5ccccc45)c4oc5ccccc5c34)cccc21] | Compound 125 | 6.28 g (81%) | 86% | 774.48 |
| P25 | 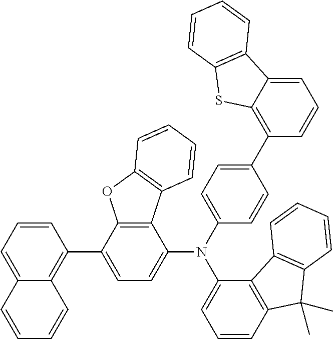 [SMILES: CC1(C)c2ccccc2-c2c(N(c3ccc(-c4cccc5c4sc4ccccc45)cc3)c3ccc(-c4cccc5ccccc45)c4oc5ccccc5c34)cccc21] | Compound 126 | 6.01 g (76%) | 87% | 790.45 |
| P26 | 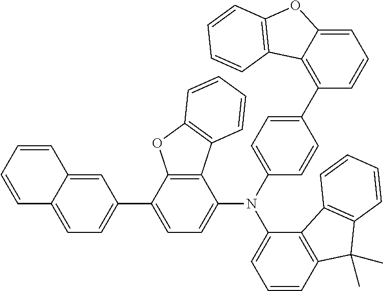 [SMILES: CC1(C)c2ccccc2-c2c(N(c3ccc(-c4cccc5oc6ccccc6c45)cc3)c3ccc(-c4ccc5ccccc5c4)c4oc5ccccc5c34)cccc21] | Compound 147 | 6.20 g (80%) | 88% | 774.48 |
| P27 | 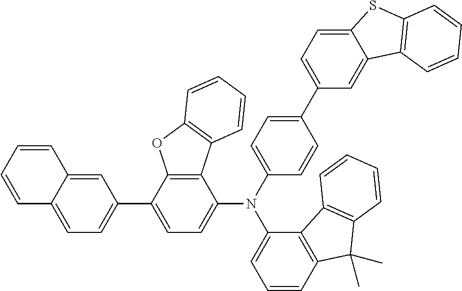 [SMILES: CC1(C)c2ccccc2-c2c(N(c3ccc(-c4ccc5sc6ccccc6c5c4)cc3)c3ccc(-c4ccc5ccccc5c4)c4oc5ccccc5c34)cccc21] | Compound 148 | 6.20 g (80%) | 87% | 790.45 |
| P28 | 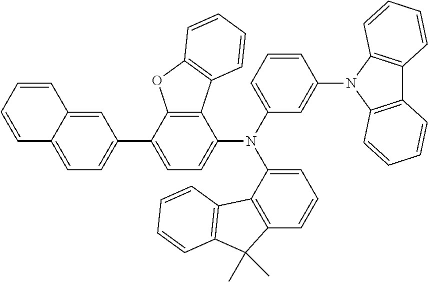 [SMILES: CC1(C)c2ccccc2-c2c(N(c3cccc(-n4c5ccccc5c5ccccc54)c3)c3ccc(-c4ccc5ccccc5c4)c4oc5ccccc5c34)cccc21] | Compound 149 | 5.85 g (74%) | 82% | 774.50 |
| P29 | 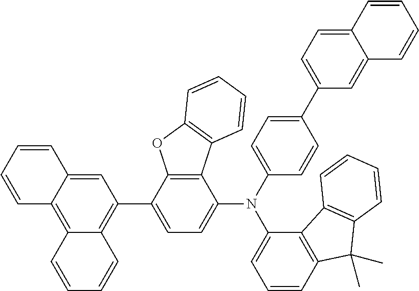 [SMILES: CC1(C)c2ccccc2-c2c(N(c3ccc(-c4ccc5ccccc5c4)cc3)c3ccc(-c4cc5ccccc5c5ccccc45)c4oc5ccccc5c34)cccc21] | Compound 168 | 6.76 g (86%) | 88% | 785.50 |
| P30 | 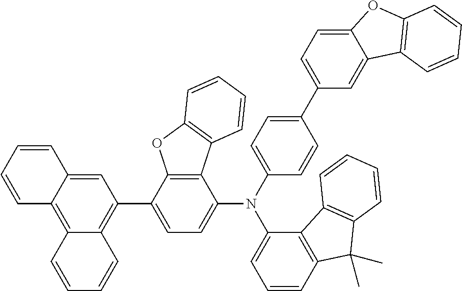 [SMILES: CC1(C)c2ccccc2-c2c(N(c3ccc(-c4ccc5oc6ccccc6c5c4)cc3)c3ccc(-c4cc5ccccc5c5ccccc45)c4oc5ccccc5c34)cccc21] | Compound 169 | 6.70 g (81%) | 82% | 826.51 |
| P31 | 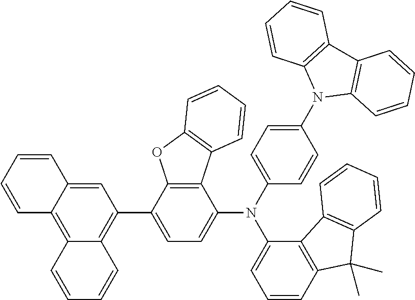 [SMILES: CC1(C)c2ccccc2-c2c(N(c3ccc(-n4c5ccccc5c5ccccc54)cc3)c3ccc(-c4cc5ccccc5c5ccccc45)c4oc5ccccc5c34)cccc21] | Compound 171 | 6.37 g (77%) | 82% | 826.53 |

### [Experimental Example 1] Measurement of HOMO and LUMO

It is preferable that the hole transport auxiliary layer serves to reduce the accumulation of holes at the interface of the emitting layer due to the HOMO level difference between the hole transport layer and the emitting layer, and to this end, the HOMO energy difference with the emitting layer is smaller than the HOMO energy difference with the hole injection layer. In addition, to minimize electrons from leaking from the emitting layer to the hole transport layer, the hole transport auxiliary layer should have a higher LUMO energy level than the emitting layer.

To check whether the compound represented by Chemical Formula 1 according to the present disclosure is suitable as the material for the hole transport auxiliary layer, values (eV) of HOMO and LUMO were calculated using Spartan software (B3LYP DFT 6-31G* by spartan'16), which is shown in Table 3 below.

TABLE 3

| Compound | HOMO (calculation) | LUMO (calculation) |
|---|---|---|
| 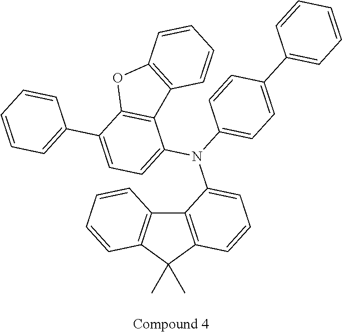 [SMILES: CC1(C)c2ccccc2-c2c(N(c3ccc(-c4ccccc4)cc3)c3ccc(-c4ccccc4)c4oc5ccccc5c34)cccc21] | −5.03 | −1.08 |
| 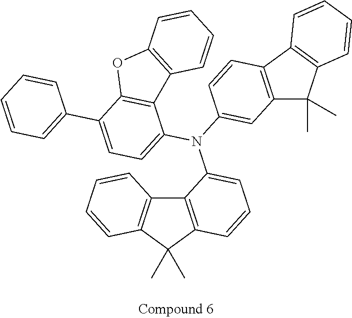 [SMILES: CC1(C)c2ccccc2-c2ccc(N(c3cccc4c3-c3ccccc3C4(C)C)c3ccc(-c4ccccc4)c4oc5ccccc5c34)cc21] | −4.90 | −1.09 |
| 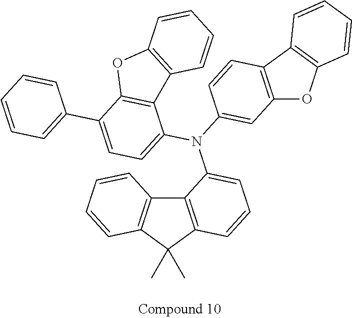 [SMILES: CC1(C)c2ccccc2-c2c(N(c3ccc4c(c3)oc3ccccc34)c3ccc(-c4ccccc4)c4oc5ccccc5c34)cccc21] | −5.03 | −1.16 |
| 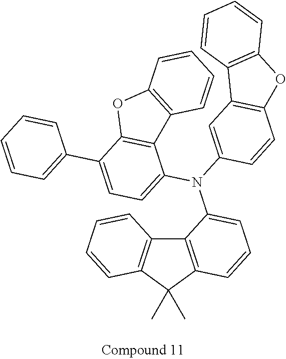 [SMILES: CC1(C)c2ccccc2-c2c(N(c3ccc4oc5ccccc5c4c3)c3ccc(-c4ccccc4)c4oc5ccccc5c34)cccc21] | −5.01 | −1.11 |

### [Example 1] Manufacturing of Organic Light Emitting Diode (Blue Emitting Layer)

A substrate on which ITO (100 nm) that was an anode of an organic light emitting diode was stacked was separately patterned as a cathode, an anode area, and an insulating layer through a photo-lithograph process, and then to increase a work function of the anode (ITO) and perform cleaning, the surface was treated with UV-ozone treatment and O₂:N₂ plasma.

Next, a mixture of NDP-9(2-(7-Dicyanomethylene-1,3,4,5,6,8,9,10-octafluoro-7H-pyren-2-ylidene)-malononitrile) and N4,N4,N4′,N4′-tetra([1,1′-biphenyl]-4-yl)-[1,1′-biphenyl]-4,4′-diamine mixed in a ratio of 3:97 as the hole injection layer (HIL) was deposited and formed in the thickness of 10 nm on the anode. Subsequently, N4,N4,N4′,N4′-tetra([1,1′-biphenyl]-4-yl)-[1,1′-biphenyl]-4,4′-diamine as the hole transport layer was deposited in vacuum and formed in the thickness of 100 nm on the hole injection layer, and Compound 4 was formed as hole transport auxiliary layer in the thickness of 15 nm on the hole transport layer (HTL).

A blue emitting layer (EML) was deposited in the thickness of 25 nm on the hole transport auxiliary layer using 9,10-Bis(2-naphthyl)anthracene (ADN) as a host and 2,12-Di-tert-butyl-5,9-bis(4-(tert-butyl)phenyl)-7-(3,5-di-tert-butylphenyl)-5,9-dihydro5,9-diaza-13b-boranaphtho[3,2,1-de]anthracene (t-DABNA-dtB) as a dopant, and a mixing ratio (based on weight) of host:dopant was 97:3. A mixture of 2-(4-(9,10-Di(naphthalene-2-yl) anthracene-2-yl)phenyl)-1-phenyl-1H-benzo[d]imidazole and LiQ mixed at a weight ratio of 1:1 as the electron transport layer (ETL) was deposited in the thickness of 25 nm on the blue emitting layer (EML). The electron injection layer (LiQ) was deposited in the thickness of 1 nm on the electrode transport layer (ETL), and a mixture of magnesium and silver mixed at a ratio of 1:4 as a cathode was deposited in the thickness of 16 nm. N4,N4′-bis[4-[bis(3-methylphenyl)amino] phenyl]-N4,N4′-diphenyl-[1,1′-biphenyl]-4,4′-diamine (DNTPD) as a capping layer was deposited in the thickness of 60 nm on the cathode. An organic light emitting diode was manufactured by bonding a seal cap containing a moisture absorbent on the capping layer using a UV curable adhesive and forming a protecting film (encapsulation layer or protecting layer) to protect the organic light emitting diode from oxygen or moisture in the atmosphere.

### Examples 2 to 31

Organic light emitting diodes of Examples 2 to 31 and Comparative Examples 1 to 5 were manufactured in the same manner as Example 1 except that the material of Compound 4 used as the material for the hole transport auxiliary layer in Example 1 was changed to ones shown in Table 4 and 5 below. The material for the hole transport auxiliary layer used in Comparative Examples 1 to 5 is as shown in Table 4 below.

TABLE 4

| | |
|---|---|
| 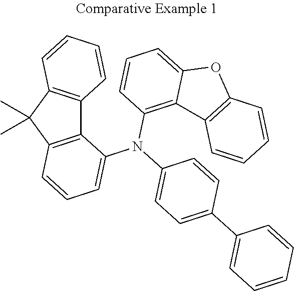 [SMILES: CC1(C)c2ccccc2-c2c(N(c3ccc(-c4ccccc4)cc3)c3cccc4oc5ccccc5c34)cccc21] | Compound A |
| 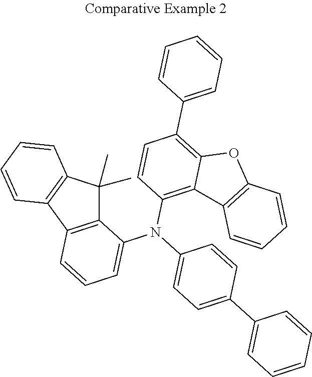 [SMILES: CC1(C)c2ccccc2-c2cccc(N(c3ccc(-c4ccccc4)cc3)c3ccc(-c4ccccc4)c4oc5ccccc5c34)c21] | Compound B |
| 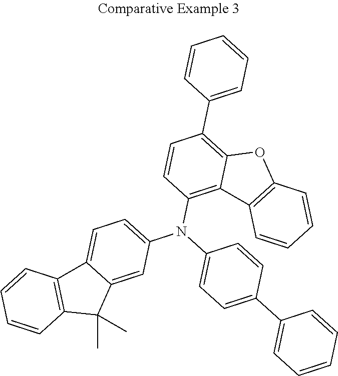 [SMILES: CC1(C)c2ccccc2-c2ccc(N(c3ccc(-c4ccccc4)cc3)c3ccc(-c4ccccc4)c4oc5ccccc5c34)cc21] | Compound C |
| 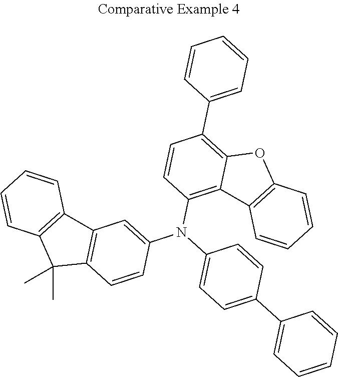 [SMILES: CC1(C)c2ccccc2-c2cc(N(c3ccc(-c4ccccc4)cc3)c3ccc(-c4ccccc4)c4oc5ccccc5c34)ccc21] | Compound D |
| 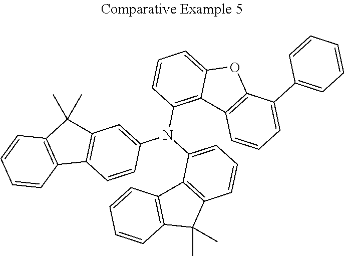 [SMILES: CC1(C)c2ccccc2-c2ccc(N(c3cccc4c3-c3ccccc3C4(C)C)c3cccc4oc5c(-c6ccccc6)cccc5c34)cc21] | Compound E |

### [Experimental Example 2] Performance Evaluation of Organic Light Emitting Diode (Blue Emitting Layer)

For each of the organic light emitting diodes manufactured in Examples 1 to 31 and Comparative Examples 1 to 5, a current of 10 mA/cm² was applied using a CS-2000 from KONICA MINOLTA to measure a driving voltage and external quantum efficiency (EQE) (%). In addition, the lifetime (LT95) was measured by a method of checking the time when a brightness decreases from an initial brightness to a 95% level by driving with a constant current of 10 mA/cm² using McScience M6000. The measurement result is shown in Table 5 below.

TABLE 5

| Example/ | Material for | Driving | | Lifetime |
|---|---|---|---|---|
| Comparative | hole transport | voltage | EQE | (LT95) |
| Example | auxiliary layer | (V) | (%) | (hrs) |
| Example 1 | Compound 4 | 3.32 | 19.5 | 386 |
| Example 2 | Compound 6 | 3.23 | 19.9 | 380 |
| Example 3 | Compound 11 | 3.40 | 19.3 | 390 |
| Example 4 | Compound 10 | 3.40 | 19.4 | 395 |
| Example 5 | Compound 85 | 3.35 | 19.8 | 382 |
| Example 6 | Compound 89 | 3.52 | 19.4 | 393 |
| Example 7 | Compound 93 | 3.53 | 19.4 | 392 |
| Example 8 | Compound 98 | 3.49 | 19.0 | 395 |
| Example 9 | Compound 101 | 3.50 | 19.2 | 397 |
| Example 10 | Compound 111 | 3.56 | 19.5 | 398 |
| Example 11 | Compound 116 | 3.59 | 19.5 | 397 |
| Example 12 | Compound 122 | 3.58 | 19.5 | 390 |
| Example 13 | Compound 128 | 3.45 | 19.7 | 384 |
| Example 14 | Compound 130 | 3.44 | 19.9 | 388 |
| Example 15 | Compound 132 | 3.55 | 19.5 | 397 |
| Example 16 | Compound 143 | 3.57 | 19.1 | 399 |
| Example 17 | Compound 150 | 3.43 | 19.9 | 387 |
| Example 18 | Compound 162 | 3.56 | 19.3 | 398 |
| Example 19 | Compound 165 | 3.54 | 19.5 | 399 |
| Example 20 | Compound 103 | 3.58 | 19.5 | 413 |
| Example 21 | Compound 104 | 3.55 | 19.6 | 410 |
| Example 22 | Compound 105 | 3.56 | 19.9 | 417 |
| Example 23 | Compound 124 | 3.55 | 19.9 | 419 |
| Example 24 | Compound 125 | 3.49 | 19.8 | 415 |
| Example 25 | Compound 126 | 3.52 | 19.8 | 413 |
| Example 26 | Compound 147 | 3.45 | 19.9 | 416 |
| Example 27 | Compound 148 | 3.52 | 19.7 | 413 |
| Example 28 | Compound 149 | 3.55 | 19.9 | 416 |
| Example 29 | Compound 168 | 3.35 | 19.9 | 408 |
| Example 30 | Compound 169 | 3.43 | 19.6 | 418 |
| Example 31 | Compound 171 | 3.55 | 19.9 | 419 |
| Comparative | Compound A | 3.88 | 18.0 | 290 |
| Example 1 | | | | |
| Comparative | Compound B | 3.89 | 17.8 | 215 |
| Example 2 | | | | |
| Comparative | Compound C | 3.87 | 17.9 | 204 |
| Example 3 | | | | |
| Comparative | Compound D | 3.73 | 16.9 | 200 |
| Example 4 | | | | |
| Comparative | Compound E | 3.72 | 16.4 | 215 |
| Example 5 | | | | |

### [Example 32] Manufacturing of Organic Light Emitting Diode (Green Emitting Layer)

A substrate on which ITO (100 nm) that was an anode of an organic light emitting diode was stacked was separately patterned as a cathode, an anode area, and an insulating layer through a photo-lithograph process, and then to increase a work function of the anode (ITO) and perform cleaning, the surface was treated with UV-ozone treatment and O₂:N₂ plasma.

Next, a mixture of NDP-9(2-(7-Dicyanomethylene-1,3,4,5,6,8,9,10-octafluoro-7H-pyren-2-ylidene)-malononitrile) and N4,N4,N4′,N4′-tetra([1,1′-biphenyl]-4-yl)-[1,1′-biphenyl]-4,4′-diamine mixed in a ratio of 3:97 as the hole injection layer (HIL) was deposited and formed in the thickness of 10 nm on the anode. Subsequently, N4,N4,N4′,N4′-tetra([1,1′-biphenyl]-4-yl)-[1,1′-biphenyl]-4,4′-diamine as the hole transport layer was deposited in vacuum and formed in the thickness of 100 nm on the hole injection layer, and Compound 4 was formed as hole transport auxiliary layer in the thickness of 15 nm on the hole transport layer (HTL).

A green lighting layer (EML) was deposited in the thickness of 35 nm on the hole transport auxiliary layer using 4,4′-N,N′-dicarbazole-biphenyl (CBP) as a host and Ir(ppy)₃[tris(2-phenylpyridine)-iridium] as a dopant, and a mixing ratio (based on weight) of host:dopant was 95:5. A mixture of 2-(4-(9,10-Di(naphthalene-2-yl)anthracene-2-yl)phenyl)-1-phenyl-1H-benzo[d]imidazole and LiQ mixed at a weight ratio of 1:1 as the electron transport layer (ETL) was deposited in the thickness of 25 nm on the green emitting layer (EML). LiQ as the electron injection layer (EIL) was deposited in the thickness of 1 nm on the electron transport layer (ETL). Then, a mixture mixing magnesium and silver (Ag) at 1:4 was deposited in a thickness of 16 nm as a negative electrode, and N4,N4′-bis[4-[bis(3-Methylphenyl)amino]phenyl]-N4,N4′-diphenyl-[1,1′-biphenyl]-4,4′-diamine (DNTPD) as a capping layer was deposited above the negative electrode in a thickness of 60 nm. An organic light emitting diode was manufactured by bonding a seal cap containing a moisture absorbent on the capping layer using a UV curable adhesive and forming a protecting film (encapsulation layer or protecting layer) to protect the organic light emitting diode from oxygen or moisture in the atmosphere.

### Exanples 33 to 62

Organic light emitting diodes of Examples 33 to 62 and Comparative Examples 6 to 10 were manufactured in the same manner as Example 32 except that the material of Compound 4 used as the material for the hole transport auxiliary layer in Example 32 was changed to one shown in Table 6 below. Structures of Compounds A to E that are materials for the hole transport auxiliary layer used in Comparative Examples 6 to 10 are the same as ones shown in Table 4.

### [Experimental Exanple 3] Performance Evaluation of Organic Light Emitting Diode (Green Emitting Layer)

For each of the organic light emitting diodes manufactured in Examples 32 to 62 and Comparative Examples 6 to 10, a current of 10 mA/cm² was applied using a CS-2000 from KONICA MINOLTA to measure a driving voltage and external quantum efficiency (EQE) (%). In addition, the lifetime (LT95) was measured by a method of checking the time when a brightness decreases from an initial brightness to a 95% level by driving with a constant current of 10 mA/cm² using McScience M6000. The measurement result is shown in Table 6 below.

TABLE 6

| Example/ | Material for | Driving | | Lifetime |
|---|---|---|---|---|
| Comparative | hole transport | voltage | EQE | (LT95) |
| Example | auxiliary layer | (V) | (%) | (hrs) |
| Example 32 | Compound 4 | 3.67 | 38.5 | 189 |
| Example 33 | Compound 6 | 3.63 | 39.5 | 177 |
| Example 34 | Compound 11 | 3.76 | 37.7 | 198 |
| Example 35 | Compound 10 | 3.76 | 37.8 | 199 |
| Example 36 | Compound 85 | 3.70 | 38.6 | 183 |
| Example 37 | Compound 89 | 3.77 | 37.5 | 202 |
| Example 38 | Compound 93 | 3.78 | 37.7 | 195 |
| Example 39 | Compound 98 | 3.75 | 37.9 | 200 |
| Example 40 | Compound 101 | 3.75 | 38.0 | 199 |
| Example 41 | Compound 111 | 3.76 | 38.0 | 199 |
| Example 42 | Compound 116 | 3.79 | 37.9 | 198 |
| Example 43 | Compound 122 | 3.79 | 37.9 | 201 |
| Example 44 | Compound 128 | 3.74 | 38.5 | 185 |
| Example 45 | Compound 130 | 3.75 | 38.8 | 188 |
| Example 46 | Compound 132 | 3.77 | 37.6 | 195 |
| Example 47 | Compound 143 | 3.75 | 38.0 | 198 |
| Example 48 | Compound 150 | 3.68 | 38.6 | 188 |
| Example 49 | Compound 162 | 3.76 | 38.0 | 198 |
| Example 50 | Compound 165 | 3.79 | 37.9 | 199 |
| Example 51 | Compound 103 | 3.76 | 38.6 | 215 |
| Example 52 | Compound 104 | 3.75 | 38.6 | 213 |
| Example 53 | Compound 105 | 3.75 | 38.4 | 216 |
| Example 54 | Compound 124 | 3.74 | 39.0 | 215 |
| Example 55 | Compound 125 | 3.81 | 38.8 | 213 |
| Example 56 | Compound 126 | 3.74 | 38.7 | 212 |
| Example 57 | Compound 147 | 3.77 | 39.0 | 213 |
| Example 58 | Compound 148 | 3.79 | 38.9 | 215 |
| Example 59 | Compound 149 | 3.75 | 39.1 | 218 |
| Example 60 | Compound 168 | 3.76 | 39.5 | 210 |
| Example 61 | Compound 169 | 3.75 | 39.2 | 217 |
| Example 62 | Compound 171 | 3.76 | 39.2 | 215 |
| Comparative | Compound A | 4.19 | 30.3 | 110 |
| Example 6 | | | | |
| Comparative | Compound B | 4.26 | 31.1 | 92 |
| Example 7 | | | | |
| Comparative | Compound C | 4.22 | 27.0 | 84 |
| Example 8 | | | | |
| Comparative | Compound D | 4.25 | 33.3 | 103 |
| Example 9 | | | | |
| Comparative | Compound E | 4.20 | 34.4 | 55 |
| Example 10 | | | | |

According to the experimental results in Tables 5 and 6, it was confirmed that when the compound of the present disclosure was used as the material for the hole transport auxiliary layer of the organic light emitting diode, the driving voltage was low, and excellent element efficiency characteristics and long lifetime characteristics were shown compared to the compounds in Comparative Examples.

In other words, it was confirmed that the compounds of the present disclosure had the fourth position of the dimethylfluorene group and the first position of the dibenzofuran group in the compound represented by Chemical Formula 1, which had the form bonded to the nitrogen (N) of the amine group as the single bond, and had the suitable energy level (reduction in HOMO energy level difference between the hole transport layer and the emitting layer) as the hole transport auxiliary layer serving to transfer holes from the hole transport layer to the emitting layer and block electrons coming from the emitting layer, thereby showing excellent characteristics as the material for the hole transport auxiliary layer.

In addition, since the compounds of the present disclosure have the above-described characteristic structural forms, the compounds may adjust hole injection characteristics compared to the compounds in Comparative Examples to reduce the accumulation of holes at the interface between the hole transport auxiliary layer and the emitting layer, thereby minimizing the quenching phenomenon in which excitons are annihilated by polarons at the interface between the hole transport auxiliary layer and the emitting layer. As a result, it was confirmed that it was possible to minimize the degradation phenomenon of the element and stabilize the element compared to the compounds in Comparative Examples, thereby reducing the driving voltage and increasing efficiency and lifetime when the compounds of the present disclosure was applied to the element.

Although the embodiments of the present disclosure have been described above in detail, the scope of the present disclosure is not limited thereto, and various modifications and improvements of those skilled in the art using the basic concept of the present disclosure defined in the appended claims are also included in the scope of the present disclosure.

What is claimed is:

1. An organic compound represented by a following Chemical Formula 1: 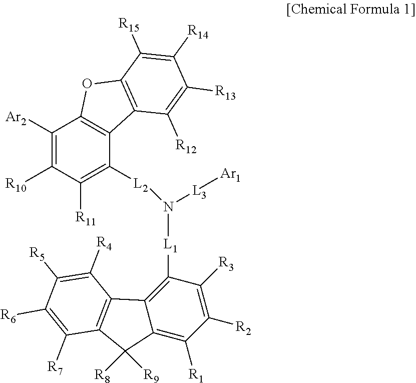
in Chemical Formula 1,
L₁ to L₃ are, each independently, one selected from the group consisting of a single bond; a substituted or unsubstituted aryl group having 6 to 30 carbon atoms; a substituted or unsubstituted aralkyl group having 7 to 30 carbon atoms; a substituted or unsubstituted heteroaryl group having 5 to 60 carbon atoms; and a substituted or unsubstituted heteroarylalkyl group having 6 to 60 carbon atoms,
R₁ to R₁₅, Ar₁ and Ar₂ are, each independently, one selected from the group consisting of hydrogen; deuterium; a cyano group; a nitro group; halogen; a hydroxy group; a substituted or unsubstituted alkyl group having 1 to 30 carbon atoms; a substituted or unsubstituted alkenyl group having 2 to 30 carbon atoms; a substituted or unsubstituted alkynyl group having 2 to 24 carbon atoms; a substituted or unsubstituted heteroalkyl group having 2 to 30 carbon atoms; a substituted or unsubstituted aryl group having 6 to 30 carbon atoms; a substituted or unsubstituted heteroaryl group having 2 to 30 carbon atoms; a substituted or unsubstituted heteroarylalkyl group having 3 to 30 carbon atoms; a substituted or unsubstituted aralkyl group having 6 to 30 carbon atoms; a substituted or unsubstituted cycloalkyl group having 3 to 20 carbon atoms; a substituted or unsubstituted heterocycloalkyl group having 3 to 20 carbon atoms; a substituted or unsubstituted alkoxy group having 1 to 30 carbon atoms; a substituted or unsubstituted alkylsilyl group having 1 to 30 carbon atoms; a substituted or unsubstituted arylsilyl group having 6 to 30 carbon atoms; and a substituted or unsubstituted aryloxy group having 6 to 30 carbon atoms, and
optionally, when L₁ to L₃, R₁ to R₁₅, and Ar₁ and Ar₂ are substituted, substituents are one or more selected from the group consisting of deuterium; a cyano group; a nitro group; halogen; a hydroxy group; an alkyl group having 1 to 30 carbon atoms; an alkenyl group having 2 to 30 carbon atoms; an alkynyl group having 2 to 24 carbon atoms; a heteroalkyl group having 2 to 30 carbon atoms; an aralkyl group having 6 to 30 carbon atoms; a cycloalkyl group having 3 to 20 carbon atoms; a heterocycloalkyl group having 3 to 20 carbon atoms; an aryl group having 6 to 30 carbon atoms; a heteroaryl group having 2 to 30 carbon atoms; a heteroarylalkyl group having 3 to 30 carbon atoms; an alkoxy group having 1 to 30 carbon atoms; an alkylsilyl group having 1 to 30 carbon atoms; an arylsilyl group having 6 to 30 carbon atoms; and an aryloxy group having 6 to 30 carbon atoms, and when a plurality of substituents are present, each substituent is the same as or different from each other.

2. The organic compound of claim 1, wherein L₁ and L₂ are each selected as a single bond.

3. The organic compound of claim 1, wherein L₃ is selected from the group consisting of a single bond; a substituted or unsubstituted aryl group having 6 to 30 carbon atoms; and a substituted or unsubstituted heteroaryl group having 2 to 30 carbon atoms.

4. The organic compound of claim 1, wherein Ar₁ is selected from the group consisting of a substituted or unsubstituted aryl group having 6 to 30 carbon atoms; a substituted or unsubstituted heteroaryl group having 2 to 30 carbon atoms; and a substituted or unsubstituted aralkyl group having 6 to 30 carbon atoms.

5. The organic compound of claim 1, wherein Ar₂ is selected as a substituted or unsubstituted aryl group having 6 to 30 carbon atoms.

6. The organic compound of claim 1, wherein Chemical Formula 1 is represented by one selected from a group consisting of Chemical Formulas 2 to 5 below: 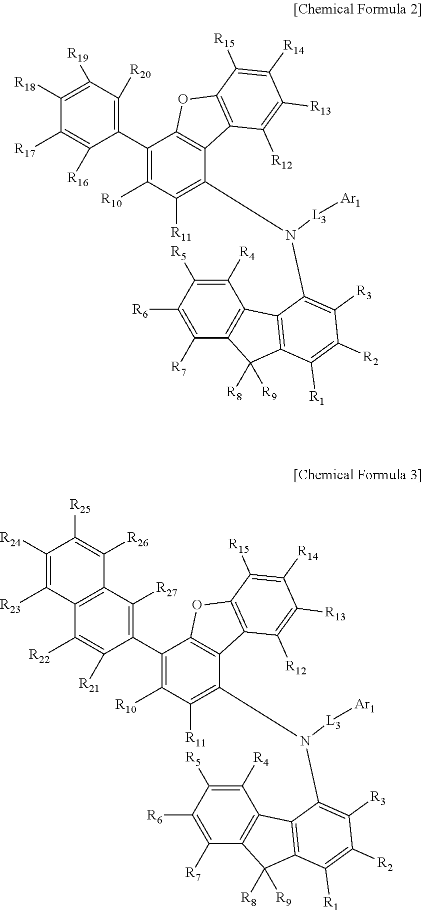 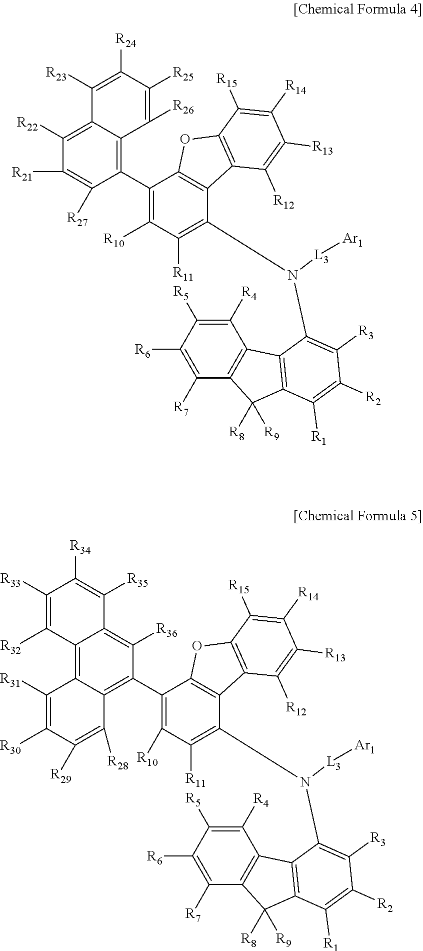
in Chemical Formulas 2 to 5,
R₁ to R₁₅, L₃, and Ar₁ are as defined in Chemical Formula 1 of claim 1,
R₁₆ to R₃₆ are, each independently, one selected from the group consisting of hydrogen; deuterium; a cyano group; a nitro group; halogen; a hydroxy group; a substituted or unsubstituted alkyl group having 1 to 30 carbon atoms; a substituted or unsubstituted alkenyl group having 2 to 30 carbon atoms; a substituted or unsubstituted alkynyl group having 2 to 24 carbon atoms; a substituted or unsubstituted heteroalkyl group having 2 to 30 carbon atoms; a substituted or unsubstituted aryl group having 6 to 30 carbon atoms; a substituted or unsubstituted heteroaryl group having 2 to 30 carbon atoms; a substituted or unsubstituted heteroarylalkyl group having 3 to 30 carbon atoms; a substituted or unsubstituted aralkyl group having 6 to 30 carbon atoms; a substituted or unsubstituted cycloalkyl group having 3 to 20 carbon atoms; a substituted or unsubstituted heterocycloalkyl group having 3 to 20 carbon atoms; a substituted or unsubstituted alkoxy group having 1 to 30 carbon atoms; a substituted or unsubstituted alkylsilyl group having 1 to 30 carbon atoms; a substituted or unsubstituted arylsilyl group having 6 to 30 carbon atoms; and a substituted or unsubstituted aryloxy group having 6 to 30 carbon atoms, and
optionally, when R₁₆ to R₃₆ are substituted, substituents are one or more selected from the group consisting of deuterium; a cyano group; a nitro group, halogen; a hydroxy group; an alkyl group having 1 to 30 carbon atoms; an alkenyl group having 2 to 30 carbon atoms; an alkynyl group having 2 to 24 carbon atoms; a heteroalkyl group having 2 to 30 carbon atoms; an aralkyl group having 6 to 30 carbon atoms; a cycloalkyl group having 3 to 20 carbon atoms; a heterocycloalkyl group having 3 to 20 carbon atoms; an aryl group having 6 to 30 carbon atoms; a heteroaryl group having 2 to 30 carbon atoms; a heteroarylalkyl group having 3 to 30 carbon atoms; an alkoxy group having 1 to 30 carbon atoms; an alkylsilyl group having 1 to 30 carbon atoms; an arylsilyl group having 6 to 30 carbon atoms; and an aryloxy group having 6 to 30 carbon atoms, and when a plurality of substituents are present, each substituent is the same as or different from each other.

7. The organic compound of claim 1, wherein Chemical Formula 1 is represented by one selected from a group consisting of Chemical Formulas 6 to 9 below: 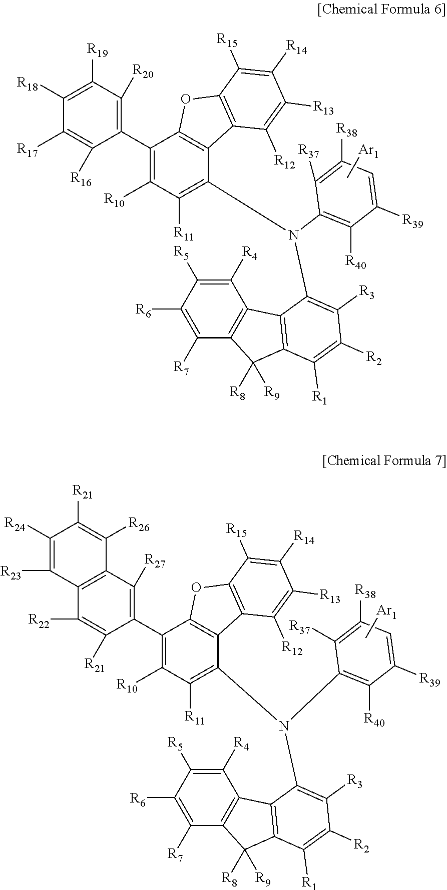 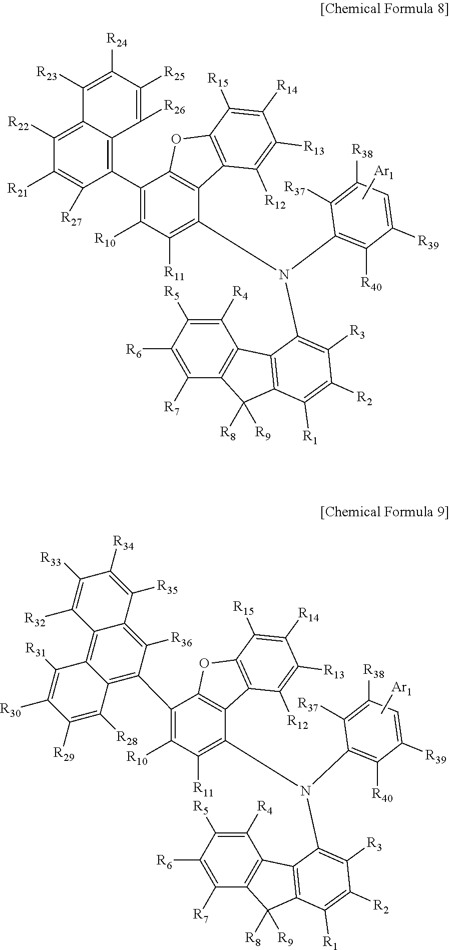
in Chemical Formulas 6 to 9,
R₁ to R₁₅ and Ar₁ are as defined in Chemical Formula 1 of claim 1,
R₁₆ to R₄₀ are, each independently, one selected from the group consisting of hydrogen; deuterium; a cyano group; a nitro group; halogen; a hydroxy group; a substituted or unsubstituted alkyl group having 1 to 30 carbon atoms; a substituted or unsubstituted alkenyl group having 2 to 30 carbon atoms; a substituted or unsubstituted alkynyl group having 2 to 24 carbon atoms; a substituted or unsubstituted heteroalkyl group having 2 to 30 carbon atoms; a substituted or unsubstituted aryl group having 6 to 30 carbon atoms; a substituted or unsubstituted heteroaryl group having 2 to 30 carbon atoms; a substituted or unsubstituted heteroarylalkyl group having 3 to 30 carbon atoms; a substituted or unsubstituted aralkyl group having 6 to 30 carbon atoms; a substituted or unsubstituted cycloalkyl group having 3 to 20 carbon atoms; a substituted or unsubstituted heterocycloalkyl group having 3 to 20 carbon atoms; a substituted or unsubstituted alkoxy group having 1 to 30 carbon atoms; a substituted or unsubstituted alkylsilyl group having 1 to 30 carbon atoms; a substituted or unsubstituted arylsilyl group having 6 to 30 carbon atoms; and a substituted or unsubstituted aryloxy group having 6 to 30 carbon atoms, and
optionally, when R₁₆ to R₄₀ are substituted, substituents are one or more selected from the group consisting of deuterium; a cyano group; a nitro group; halogen; a hydroxy group; an alkyl group having 1 to 30 carbon atoms; an alkenyl group having 2 to 30 carbon atoms; an alkynyl group having 2 to 24 carbon atoms; a heteroalkyl group having 2 to 30 carbon atoms; an aralkyl group having 6 to 30 carbon atoms; a cycloalkyl group having 3 to 20 carbon atoms; a heterocycloalkyl group having 3 to 20 carbon atoms; an aryl group having 6 to 30 carbon atoms; a heteroaryl group having 2 to 30 carbon atoms; a heteroarylalkyl group having 3 to 30 carbon atoms, an alkoxy group having 1 to 30 carbon atoms; an alkylsilyl group having 1 to 30 carbon atoms; an arylsilyl group having 6 to 30 carbon atoms; and an aryloxy group having 6 to 30 carbon atoms, and when a plurality of substituents are present, each substituent is the same as or different from each other.

8. An organic light emitting diode comprising:
an anode;
a cathode facing the anode; and
one or more organic layer between the anode and the cathode,
wherein at least one of the organic layers includes the organic compound of claim 1.

9. The organic light emitting diode of claim 8, wherein the organic layer including the organic compound of claim 1 is a hole transport auxiliary layer.

10. The organic light emitting diode of claim 8, wherein the organic layer further includes one or more selected from the group consisting of a hole injection layer, a hole transport layer, an emitting layer, an electron transport layer, and an electron injection layer.
